SEQUENCE LISTING (188 sequences)

SEQ ID NO: 1-108 - skipped (108 entries)

SEQ ID NO: 109 - protein, 124 aa, Homo sapiens
EVQLVESGGGLVKPGDSLTVSCVASGFTFSNYFIHWVRQAPGKGLEWVATISGRTKYMFYSDSLRGRFTVSRDNANNSVYLHMSSLRGEDTALYYCVRGYDHSDSNSAADLLHWGRGTLVTVSS

SEQ ID NO: 110 - protein, 124 aa, Homo sapiens
EVQLVESGGGLVKPGGSLRLSCAASGFPFSRYSMHWVRQAPGKGLEWVSSMTSRTNHKYYADSLKGRFLISRDNDRDSLYLEMNSLGVEDTAIYFCARGYDTSGSDSGVDFQYWGQGTLVTVSS

SEQ ID NO: 111-112 - skipped (2 entries)

SEQ ID NO: 113 - protein, 111 aa, Homo sapiens
NFMLTQPLSVSGYPGKTIVISCVRSSGSVASHYVQWFQQRPGSAPAMVIYEDSHRPSGIPDRFSGSVDASSNSASLTISGLKTEDEADYFCQSYYGNNQVLFGGGTKLTVL

SEQ ID NO: 114 - protein, 111 aa, Homo sapiens
NFMLTQPHSVSESPGKTITISCTRGRGYIASYYVQWYQQRPGGVPKIVVFEDTQRPSGVPDRISGSIDTSTNSASLTISGLQTEDEADYYCQSYDDANHVIFGGGTKLTVL

SEQ ID NO: 115-119 - skipped (5 entries)

SEQ ID NO: 120 - protein, 8 aa, Homo sapiens
GFTFSNYF

SEQ ID NO: 121 - protein, 8 aa, Homo sapiens
ISGRTKYM

SEQ ID NO: 122 - protein, 17 aa, Homo sapiens
VRGYDHSDSNSAADLLH

SEQ ID NO: 123 - protein, 8 aa, Homo sapiens
GFPFSRYS

SEQ ID NO: 124 - protein, 8 aa, Homo sapiens
MTSRTNHK

SEQ ID NO: 125 - protein, 17 aa, Homo sapiens
ARGYDTSGSDSGVDFQY

SEQ ID NO: 126-131 - skipped (6 entries)

SEQ ID NO: 132 - protein, 8 aa, Homo sapiens
SGSVASHY

SEQ ID NO: 133 - skipped

SEQ ID NO: 134 - protein, 10 aa, Homo sapiens
QSYYGNNQVL

SEQ ID NO: 135 - protein, 8 aa, Homo sapiens
RGYIASYY

SEQ ID NO: 136 - skipped

SEQ ID NO: 137 - protein, 10 aa, Homo sapiens
QSYDDANHVI

SEQ ID NO: 138-143 - skipped (6 entries)

SEQ ID NO: 144 - protein, 10 aa, Homo sapiens
GFTFSNYFIH

SEQ ID NO: 145 - protein, 17 aa, Homo sapiens
TISGRTKYMFYSDSLRG

SEQ ID NO: 146 - protein, 15 aa, Homo sapiens
GYDHSDSNSAADLLH

SEQ ID NO: 147 - protein, 10 aa, Homo sapiens
GFPFSRYSMH

SEQ ID NO: 148 - protein, 17 aa, Homo sapiens
SMTSRTNHKYYADSLKG

SEQ ID NO: 149 - protein, 15 aa, Homo sapiens
GYDTSGSDSGVDFQY

SEQ ID NO: 150-155 - skipped (6 entries)

SEQ ID NO: 156 - protein, 13 aa, Homo sapiens
VRSSGSVASHYVQ

SEQ ID NO: 157 - protein, 7 aa, Homo sapiens
EDSHRPS

SEQ ID NO: 158 - protein, 13 aa, Homo sapiens
TRGRGYIASYYVQ

SEQ ID NO: 159 - protein, 7 aa, Homo sapiens
EDTQRPS

SEQ ID NO: 160-163 - skipped (4 entries)

SEQ ID NO: 164 - protein, 124 aa, synthetic construct
  REGION 1..124: note = Recombinant variant antibody sequence
EVQLVESGGGLVKPGDSLTVSCVASGFTFSNYFIHWVRQAPGKGLEWVATISGRTKYMFYSDSLRGRFTVSRDNAANSVYLHMSSLRGEDTALYYCVRGYDHSDSNSAADLLHWGRGTLVTVSS

SEQ ID NO: 165 - protein, 124 aa, synthetic construct
  REGION 1..124: note = Recombinant variant antibody sequence
EVQLVESGGGLVKPGDSLTVSCVASGFTFSNYFIHWVRQAPGKGLEWVATISGRTKYMFYSDSLRGRFTVSRDNAQNSVYLHMSSLRGEDTALYYCVRGYDHSDSNSAADLLHWGRGTLVTVSS

SEQ ID NO: 166-182 - skipped (17 entries)

SEQ ID NO: 183 - protein, 454 aa, Homo sapiens
EVQLVESGGGLVKPGDSLTVSCVASGFTFSNYFIHWVRQAPGKGLEWVATISGRTKYMFYSDSLRGRFTVSRDNANNSVYLHMSSLRGEDTALYYCVRGYDHSDSNSAADLLHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 217 aa, Homo sapiens
NFMLTQPLSVSGYPGKTIVISCVRSSGSVASHYVQWFQQRPGSAPAMVIYEDSHRPSGIPDRFSGSVDASSNSASLTISGLKTEDEADYFCQSYYGNNQVLFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 185 - protein, 454 aa, Homo sapiens
EVQLVESGGGLVKPGGSLRLSCAASGFPFSRYSMHWVRQAPGKGLEWVSSMTSRTNHKYYADSLKGRFLISRDNDRDSLYLEMNSLGVEDTAIYFCARGYDTSGSDSGVDFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 217 aa, Homo sapiens
NFMLTQPHSVSESPGKTITISCTRGRGYIASYYVQWYQQRPGGVPKIVVFEDTQRPSGVPDRISGSIDTSTNSASLTISGLQTEDEADYYCQSYDDANHVIFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 187 - protein, 454 aa, synthetic construct
  REGION 1..454: note = Recombinant antibody variant
EVQLVESGGGLVKPGDSLTVSCVASGFTFSNYFIHWVRQAPGKGLEWVATISGRTKYMFYSDSLRGRFTVSRDNAANSVYLHMSSLRGEDTALYYCVRGYDHSDSNSAADLLHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 454 aa, synthetic construct
  REGION 1..454: note = Recombinant antibody variant
EVQLVESGGGLVKPGDSLTVSCVASGFTFSNYFIHWVRQAPGKGLEWVATISGRTKYMFYSDSLRGRFTVSRDNAQNSVYLHMSSLRGEDTALYYCVRGYDHSDSNSAADLLHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0320288 A1
(43) Pub. Date: Oct. 16, 2025

(54) METHOD OF TREATING VITILIGO WITH INTERFERON-GAMMA ANTIBODY

(71) Applicant: Elixiron Immunotherapeutics (Hong Kong), Central Hong Kong (CN)

(72) Inventors: Hung-Kai Chen, Los Altos, CA (US); Pei-Han Chung, Taipei City (TW); Cheng-Lun KU, Taoyuan City (TW); Chau Yee Ng, Taoyuan City (TW)

(21) Appl. No.: 18/869,871

(22) PCT Filed: Jun. 2, 2023
(86) PCT No.: PCT/US2023/067809

§ 371 Date: Nov. 27, 2024

Related U.S. Application Data
(60) Provisional application No. 63/348,380, filed on Jun. 2, 2022.

(51) Int. Cl. C07K 16/24; A61K 39/00; A61P 17/00

(52) U.S. Cl. CPC C07K 16/249; A61P 17/00; A61K 2039/505; C07K 2317/76

(57) ABSTRACT

The present invention provides methods for treating vitiligo using antibodies which specifically bind to IFN-γ.

### FIELD

The present disclosure relates generally to methods for treating vitiligo, particularly methods that use antibodies that bind to interferon-gamma (IFN-γ).

### BACKGROUND

Vitiligo is a T cell-mediated inflammatory skin disorder that progressively destroys melanocytes in the skin, resulting in the loss of epidermal melanocytes, patchy disfiguring depigmentation and an unpredictable clinical course that complicates patient management. IFN-γ and its' signature cytokines, including CXCL9, CXCL10 and Granzyme B (GzmB), are most highly expressed in the skin lesion of vitiligo patients and are critical cytokines that contribute to the recruitment of autoreactive cytotoxic T cells in vitiligo patients. IFN-γ directly works on melanocytes by decreasing the viability of melanocytes, inducing apoptosis and/or downregulating melanogenesis. IFN-γ may stimulate immune cells by activating immune receptor (CXCR3) expression during T cell activation and stimulate CXCL9/CXCL10 expression. Therefore, reducing IFN-γ expression will reduce T cell trafficking to inflammatory lesion site.

IFN-γ is a crucial regulator of the host immune system and contributes to autoimmune pathology in many diseases. Aside from functioning as an inducer for chemokines to recruit T cells, previous reports showed that IFN-γ signaling maintains skin pigmentation homeostasis in a leprosy model. The direct pathological effect of IFN-γ to the melanocytes cells in vitiligo is rarely studied. There are contradictory results to which cytokine exerts the critical cytotoxic effect on melanocyte. Induced cell death, oxidative stress, and impairment in melanogenesis are critical in the pathogenesis of vitiligo. Most studies of cell death pathways in vitiligo are limited to cell apoptosis. Recent advances have led to the discovery of many new regulated cell death pathways, including necroptosis, pyroptosis, and ferroptosis, which may be responsible for the loss of melanocytes in vitiligo.

### SUMMARY

In contrast to the understanding in the art regarding IFN-γ and vitiligo, the present disclosure provides methods and compositions for treatment based upon the surprising discovery that antibodies capable of blocking the activity IFN-γ are useful in treating vitiligo. Without being bound by theory, it is proposed that highly expressed cytokines in vitiligo skin lesions caused a direct toxicity to melanocytes. Ex vivo melanocyte studies support the direct role of IFN-γ per se in melanocyte cell loss, increased oxidative stress and melanogenesis disruption. In a vitiligo patient, it is proposed that IFN-γ signaling leads to melanocyte cell loss, increased oxidative stress and melanogenesis disruption. Accordingly, the present disclosure provides methods of treatment wherein the vitiligo patient is administered an IFN-γ antibody, wherein the antibody neutralizes the effects of IFN-γ signaling, and thus regulates cell death through oxidative stress-related ferroptosis cell death, which may initiate autoimmunity in vitiligo. Moreover, the in vivo toxicology results suggest that administered the anti-IFN-γ antibody is safe without adverse effects, therefore could serve as a safer and a potentially more potent option for treatment of vitiligo.

In some embodiments, the present disclosure provides a method for treating vitiligo, comprising administering to a subject in need thereof a composition comprising a therapeutically effective amount of an IFN-γ antibody and a pharmaceutically acceptable carrier. In some embodiments, the IFN-γ antibody modulates a biological function of IFN-γ. In some embodiments, the IFN-γ antibody neutralizes IFN-γ.

In some embodiments of the method, the IFN-γ antibody inhibits, decreases, and/or fully blocks the signaling activity of IFN-γ; optionally, wherein the IFN-γ antibody inhibits, decreases, and/or fully blocks IFN-γ signaling activity by pro-human IFN-γ, mature-human IFN-γ, or truncated-human IFN-γ.

In some embodiments of the method, the IFN-γ antibody reduces one or more of: IFN-γ-dependent cytokine production; IFN-γ-dependent T cell dysfunction, IFN-γ-dependent immune tolerance; and IFN-γ-dependent inflammation.

In some embodiments of the method, wherein the IFN-γ antibody reduces expression of IFN-γ, CXCL9, CXCL10 and/or CXCL11 in melanocytes.

In some embodiments of the method, the IFN-γ antibody comprises:
a VH region having a CDR-H1 amino acid sequence selected from SEQ ID NO: 120 or 123, a CDR-H2 amino acid sequence selected from SEQ ID NO: 121 or 124, and a CDR-H3 amino acid sequence selected from SEQ ID NO: 122 or 125; and
a VL region comprising a CDR-L1 amino acid sequence selected from SEQ ID NO: 132 or 135, a CDR-L2 amino acid sequence selected from SEQ ID NO: 133 or 136, and a CDR-L3 amino acid sequence selected from SEQ ID NO: 134 or 137.

In some embodiments of the method, the IFN-γ antibody comprises:
(a) a VH region having a CDR-H1 amino acid sequence of SEQ ID NO: 120, a CDR-H2 amino acid sequence of SEQ ID NO: 121, and a CDR-H3 amino acid sequence of SEQ ID NO: 122, and a VL region comprising a CDR-L1 amino acid sequence of SEQ ID NO: 132, a CDR-L2 amino acid sequence of SEQ ID NO: 133, and a CDR-L3 amino acid sequence of SEQ ID NO: 134;
(b) a VH region having a CDR-H1 amino acid sequence of SEQ ID NO: 123, a CDR-H2 amino acid sequence of SEQ ID NO: 124, and a CDR-H3 amino acid sequence of SEQ ID NO: 125, and a VL region comprising a CDR-L1 amino acid sequence of SEQ ID NO: 135, a CDR-L2 amino acid sequence of SEQ ID NO: 136, and a CDR-L3 amino acid sequence of SEQ ID NO: 137; or
(c) a VH region having a CDR-H1 amino acid sequence of SEQ ID NO: 123, a CDR-H2 amino acid sequence of SEQ ID NO: 124, and a CDR-H3 amino acid sequence of SEQ ID NO: 125, and a VL region comprising a CDR-L1 amino acid sequence of SEQ ID NO: 132, a CDR-L2 amino acid sequence of SEQ ID NO: 133, and a CDR-L3 amino acid sequence of SEQ ID NO: 134.

In some embodiments of the method, the IFN-γ antibody comprises:
a VH region having a CDR-H1 amino acid sequence selected from SEQ ID NO: 120 or 123, a CDR-H2 amino acid sequence selected from SEQ ID NO: 121 or 124, and a CDR-H3 amino acid sequence selected from SEQ ID NO: 122 or 125; and a VL region comprising a CDR-L1 amino acid sequence selected from SEQ ID NO: 132 or 135, a CDR-L2 amino acid sequence selected from SEQ ID NO: 133 or 136, and a CDR-L3 amino acid sequence selected from SEQ ID NO: 134 or 137; and
a VH region comprising an amino acid sequence having at least 90% identity to SEQ ID NO: 109, 110, 164, or 165; and a VL region comprising an amino acid sequence having at least 90% identity to SEQ ID NO: 113, or 114.

In some embodiments of the method, the IFN-γ antibody comprises:
(a) a VH region amino acid sequence of SEQ ID NO: 109, and a VL region amino acid sequence of SEQ ID NO: 113;
(b) a VH region amino acid sequence of SEQ ID NO: 110, and a VL region amino acid sequence of SEQ ID NO: 114;
(c) a VH region amino acid sequence of SEQ ID NO: 109, and a VL region amino acid sequence of SEQ ID NO: 114;
(d) a VH region amino acid sequence of SEQ ID NO: 110, and a VL region amino acid sequence of SEQ ID NO: 113;
(e) a VH region amino acid sequence of SEQ ID NO: 164, and a VL region amino acid sequence of SEQ ID NO: 113; or
(f) a VH region amino acid sequence of SEQ ID NO: 165, and a VL region amino acid sequence of SEQ ID NO: 113.

In some embodiments of the method, the IFN-γ antibody comprises:
a VH region having a CDR-H1 amino acid sequence selected from SEQ ID NO: 120 or 123, a CDR-H2 amino acid sequence selected from SEQ ID NO: 121 or 124, and a CDR-H3 amino acid sequence selected from SEQ ID NO: 122 or 125; and
a VL region comprising a CDR-L1 amino acid sequence selected from SEQ ID NO: 132 or 135, a CDR-L2 amino acid sequence selected from SEQ ID NO: 133 or 136, and a CDR-L3 amino acid sequence selected from SEQ ID NO: 134 or 137.

In some embodiments of the method, the IFN-γ antibody comprises:
a heavy chain amino acid sequence having at least 90% identity to SEQ ID NO: 183, 185, 187, or 189; and
a light chain amino acid sequence having at least 90% identity to SEQ ID NO: 184, or 186.

In some embodiments of the method, the IFN-γ antibody comprises:
(a) a heavy chain amino acid sequence of SEQ ID NO: 183, and a light chain amino acid sequence of SEQ ID NO: 184;
(b) a heavy chain amino acid sequence of SEQ ID NO: 185, and a light chain amino acid sequence of SEQ ID NO: 186;
(c) a heavy chain amino acid sequence of SEQ ID NO: 183, and a light chain amino acid sequence of SEQ ID NO: 186;
(d) a heavy chain amino acid sequence of SEQ ID NO: 185, and a light chain amino acid sequence of SEQ ID NO: 184;
(e) a heavy chain amino acid sequence of SEQ ID NO: 187, and a light chain amino acid sequence of SEQ ID NO: 184; or
(f) a heavy chain amino acid sequence of SEQ ID NO: 189, and a light chain amino acid sequence of SEQ ID NO: 184.

In some embodiments of the method, the VH region of the IFN-γ antibody further comprises an amino acid substitution selected from N76A and N76Q.

In some embodiments of the method, the IFN-γ antibody is an antibody selected from the group consisting of 2A6, 2B6, 2A6A, 2A6Q, AB, BA, AMG811, NI-0501 and Fontolizumab. In some preferred embodiments of the method, the IFN-γ antibody is 2A6, 2B6, 2A6A, 2A6Q, AB or BA. In some embodiments of the method, 2A6Q does not induce an infection or a drug induced adverse effect in an in vivo toxicology assay.

In some embodiments of the method, the IFN-γ antibody is an immunoglobulin molecule, an Fv, a disulfide linked Fv, a monoclonal antibody, an scFv, a chimeric antibody, a single domain antibody, a CDR-grafted antibody, a diabody, a human antibody, a humanized antibody, a multispecific antibody, an Fab, a dual specific antibody, an Fab′ fragment, a bispecific antibody, an F(ab′)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody, a dAb fragment, an isolated complementarity determining region (CDR), or a single chain antibody.

In some embodiments of the method, the administering to the subject is by at least one mode selected from the group consisting of: parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

In some embodiments of the method, the subject in need is a patient with vitiligo.

In some embodiments of the method, the composition is administered to the subject more than once a day, at least once a day, at least once a week, or at least once a month.

In some embodiments of the method, further comprising administering at least one additional therapeutic agent.

In some embodiments of the method, the additional therapeutic agent is administered to the subject in need before administration of the composition, after administration of the composition, and/or at the same time as the composition.

In some embodiments of the method, the IFN-γ antibody binds to human IFN-γ with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to human IFN-γ. In some embodiments, the IFN-γ antibody also binds to rhesus macaque/cynomolgus monkey IFN-γ with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to a cynomolgus IFN-γ.

In some embodiments of the method, the IFN-γ antibody increases IL-2 production from SEB-stimulated human PBMCs by at least 1.2-fold, 1.3-fold, 1.4-fold, 1.5-fold, 1.6-fold, 1.7-fold, 1.8-fold, at least 1.9-fold, at least 2-fold, at least 2.1-fold, or at least 2.20-fold.

In some embodiments, the present disclosure provides a use of a composition comprising a therapeutically effective amount of an IFN-γ antibody and a pharmaceutically acceptable carrier for treating vitiligo in a subject in need thereof.

In some embodiments, the present disclosure provides a use of a composition comprising a therapeutically effective amount of an IFN-γ antibody and a pharmaceutically acceptable carrier for manufacture of a medicament for treating vitiligo in a subject in need thereof.

In some embodiments of the use, the composition is for use with at least one additional therapeutic agent.

In some embodiments of the use, the composition is for administration by at least one mode selected from the group consisting of: parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

In some embodiments of the use, the composition is for use more than once a day, at least once a day, at least once a week, or at least once a month.

In some embodiments of the use, the IFN-γ antibody is an immunoglobulin molecule, an Fv, a disulfide linked Fv, a monoclonal antibody, an scFv, a chimeric antibody, a single domain antibody, a CDR-grafted antibody, a diabody, a human antibody, a humanized antibody, a multispecific antibody, an Fab, a dual specific antibody, an Fab′ fragment, a bispecific antibody, an F(ab′)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody, a dAb fragment, an isolated complementarity determining region (CDR), or a single chain antibody.

In some embodiments of the use, the IFN-γ antibody is an antibody selected from the group consisting of 2A6, 2B6, 2A6A, 2A6Q, AB, BA, AMG811, NI-0501 and Fontolizumab

In some embodiments, the present disclosure provides a composition comprising a therapeutically effective amount of an IFN-γ antibody and a pharmaceutically acceptable carrier for use in treating vitiligo in a subject in need thereof.

In some embodiments of the composition, the IFN-γ antibody is an antibody selected from the group consisting of 2A6, 2B6, 2A6A, 2A6Q, AB, BA, AMG811, NI-0501 and Fontolizumab.

### BRIEF DESCRIPTION OF THE FIGURES

FIG. 1 illustrates cytokine and chemokine panel expression analysis of paired skin interstitial fluid analyte in vitiligo patients and healthy controls with high sensitivity mesoscale electrochemiluminescence multiplex panel (MSD). IFN-γ, CXCL9, CXCL10, CXCL11 are highly expressed in the skin lesion of vitiligo patients. (*p<0.05, ns: non-significant, Ctrl: healthy controls)

FIG. 2A illustrates IFN-γ dose-dependently decreased viability of melanocytes. Various concentrations of recombinant human IFN-γ were added to human primary melanocytes. The cells were cultured for additional 72 hrs at 37° C. and cell viability was determined using Celltiter-Glo. The calculated IC50 of IFN-γ causing 50% inhibition of melanocyte viability was 8.114 ng/mL.

FIG. 2B illustrates neutralization of IFN-γ by EI-001, i.e., 2A6Q, restored cell viability. Various concentrations of EI-001 were added to melanocytes cultured with 30 ng/ml of recombinant human IFN-γ. The cells were cultured for additional 72 hrs at 37° C. and luciferase activity was analyzed using Celltiter Glo. The calculated EC50 of EI-001 was 5.214 ng/mL.

FIG. 2C illustrates cell viability of melanocyte after treatment with IFN-γ, CXCL9, CXCL10, CXCL11, at Day 2, and Day 7. A. Individual effect: IFN-γ in both 10 ng/mL and 100 ng/ml, but not CXCL9, CXCL10, nor CXCL11, disrupts the cell viability of melanocyte B. Synergistic effect: melanocyte treated with IFN-γ, CXCL9, CXCL10, CXCL11, do not exhibit synergistic effect to the cell death in melanocyte. C. Cell morphology change: IFN-γ but not IFN-α induces morphology change of melanocyte in primary melanocytes in 7 days of treatment. Treatment dosage 2A6Q (10 μg/mL), IFN-γ (10 or 100 ng/mL), CXCL9 (10 ng/mL), CXCL10 (10 ng/mL), CXCL11 (10 ng/ml). (*p<0.01, **p<0.0001; ns: non-significant)

FIG. 3A illustrates effects of IFN-γ on melanogenesis in human primary melanocytes. Various concentrations of EI-001 were added to melanocytes cultured with 20 ng/ml of recombinant human IFN-γ. The cells were cultured for additional 7 days at 37° C. and melanin content was analyzed. The calculated IC₅₀ of IFN-γ causing 50% inhibition of melanocyte melanogenesis was 0.392 ng/mL.

FIG. 3B illustrates that neutralization of IFN-γ by EI-001 restored melanocyte melanogenesis. Various concentrations of EI-001 were added to human primary melanocytes cultured with 20 ng/ml of recombinant human IFN-γ. The cells were cultured for additional 7 days at 37° C. and melanin contents were determined using absorbance at 470 nm. The calculated EC50 of IFN-γ to restore 50% inhibition of melanocyte melanogenesis was 129.1 ng/mL.

FIG. 3C illustrates that IFN-γ disrupts melanin production in melanocytes. A. IFN-γ induced downregulation of melanin regulators (MITF, MLANA, TYRP1, DCTT, TYR, TRPM1) in primary melanocyte. (IFN-γ 100 ng/ml 24 hrs v.s Control) B. IFN-γ affects melanin content secretion with flow cytometer. C. IFN-γ cause decrease of MITF expression in primary melanocyte. Immunoblots of MITF in melanocyte treated with IFN-γ (10 or 100 ng/ml) for 8 hrs and 24 hrs. β-actin serves as control. (*p<0.05; ns: non-significant)

FIG. 4 illustrates that EI-001 inhibited CXCR3 expression on CD4+ and CD8+ T cells. Inhibitory capacity of EI-001 on the expression of CXCR3 on CD4+ (A, C, and E) and CD8+ (B, D, and F) T cells from 3 individual donors.

FIG. 5 illustrates that IFN-γ induced early phase apoptosis. A. Flow cytometry with Annexin V-7AAD show that IFN-γ significantly upregulate Annexin V but not 7AAD B. IFN-γ induced early phase apoptosis as expressed by Annexin V in dose dependent manner and 2A6Q rescue the apoptosis. (IFN-γ at 48 hrs v.s 2A6Q 10 μg/mL). (IFN-γ (10 or 100 ng/ml), *p<0.0001; ns: non-significant)

FIG. 6 illustrates that IFN-γ induced oxidative stress and lipid peroxidation in melanocytes. A. IFN-γ induces intracellular ROS. B. IFN-γ induced lipid peroxidation in melanocyte cells. C. IFN-γ induced downregulation of ferroptosis regulators (SLC7A11, SLC3A2, Ferritin, GPX4, TFR1) in primary melanocyte. (IFN-γ 100 ng/mL 24hrs v.s Control) D. Immunoblots of SLC7A11 and SLC3A2 in melanocyte treated with IFN-γ for 2 days. GADPH serves as loading control. (IFN-γ (10 or 100 ng/ml), *p<0.05, **p<0.0001; ns: non-significant)

FIG. 7 illustrates that human monoclonal anti-IFN-γ antibody (2A6Q) rescue IFN-γ toxicity to melanocytes. A. Cell death inhibitors are unable to restore the IFN-γ induced cell death. ROS scavenger (NAC), necroptosis inhibitor (NEC-1), pyroptosis inhibitor (VX-765), apoptosis inhibitor (QVDOPH). B. Ferroptosis inhibitors rescue cell viability of melanocyte at day 7 of treatment. C. 2A6Q rescue cell viability of melanocyte in a time-dependent manner, at day 2 and day 7 of treatment. D. 2A6Q rescue cell viability of melanocyte in a time-dependent manner, at day 2 and day 7 of treatment, treated with IFN-γ, CXCL9, CXCL10, and/or CXCL11. Treatment dosage 2A6Q (10 μg/mL), IFN-γ (10 ng/ml), CXCL9 (10 ng/ml), CXCL10 (10 ng/ml), CXCL11 (10 ng/ml). (*p<0.05, **p<0.001).

### DETAILED DESCRIPTION

The present disclosure provides methods of treatment and associated compositions based upon the surprising discovery that antibodies capable of blocking the signaling activity of IFN-y are useful in treating vitiligo. Accordingly, the present disclosure provides methods of treatment of vitiligo wherein a patient in need thereof is administered an IFN-γ antibody. The IFN-γ antibodies useful in the methods and compositions are capable of decreasing, inhibiting, and/or blocking IFN-γ signaling activity. As described in greater detail below, the methods of treatment and associated compositions are thus capable of stimulating and/or otherwise restoring normal immune function that can effectively treat vitiligo from the subject in need.

### Terminology and Techniques

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising,” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in Sambrook et al., Molecular Cloning-A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”); Antibody Engineering, Vols. 1 and 2, R. Kontermann and S. Dubel, eds., Springer-Verlag, Berlin and Heidelberg (2010); Monoclonal Antibodies: Methods and Protocols, V. Ossipow and N. Fischer, eds., 2nd Ed., Humana Press (2014); Therapeutic Antibodies: From Bench to Clinic, Z. An, ed., J. Wiley & Sons, Hoboken, N.J. (2009); and Phage Display, Tim Clackson and Henry B. Lowman, eds., Oxford University Press, United Kingdom (2004).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

“IFN-γ,” “IFN-g,” or “interferon-gamma,” as used herein, refers to the various forms of the dimerized soluble cytokine, interferon gamma, that is a member of the type II class of interferons, including but not limited to, the naturally occurring IFN-γ from primates (e.g., human, rhesus, and cynomolgus), rodents, various pre-and post-translational forms of IFN-γ (e.g., pro-human IFN-γ, mature-human IFN-γ, or truncated-human IFN-γ), and recombinant forms of IFN-γ.

“Vitiligo,” as used herein, refers to a chronic autoimmune disorder that causes patches of skin to lose pigment or color. Vitiligo happens when melanocytes are attacked and destroyed, causing the skin to turn a milky-white color. The cause of vitiligo is unknown, but it may be related to immune system changes, genetic factors, stress, or sun exposure.

“Antibody,” as used herein, refers to a molecule comprising one or more polypeptide chains that specifically binds to, or is immunologically reactive with, a particular antigen. Exemplary antibodies of the present disclosure include native antibodies, whole antibodies, monoclonal antibodies, polyclonal antibodies, chimeric antibodies, humanized antibodies, human antibodies, multispecific (or heteroconjugate) antibodies (e.g., bispecific antibodies), monovalent antibodies, multivalent antibodies, antigen-binding antibody fragments (e.g., Fab′, F(ab′)2, Fab, Fv, rIgG, and scFv fragments), antibody fusions, and synthetic antibodies (or antibody mimetics).

“IFN-γ antibody,” “anti-IFN-γ antibody” or “antibody that binds IFN-γ” refers to an antibody that binds IFN-γ with sufficient affinity such that the antibody is useful as a diagnostic and/or therapeutic agent in targeting IFN-y. In some embodiments, the extent of binding of an IFN-γ antibody to an unrelated, non-IFN-γ antigen is less than about 10% of the binding of the antibody to IFN-γ as measured, e.g., by a radioimmunoassay (RIA). In some embodiments, an antibody that binds to IFN-γ has a dissociation constant (Kd) of <1 μM, <100 nM, <10 nM, <1 nM, <0.1 nM, <0.01 nM, or <0.01 nM (e.g., 10-8 M or less, e.g., from 10⁻⁸ M to 10⁻¹³ M, e.g., from 10⁻⁹ M to 10⁻¹³ M). The descriptions of the PCT applications PCT/CN2018/085836 and PCT/US2019/024663 are incorporated by reference in their entirety.

“Full-length antibody,” “intact antibody,” or “whole antibody” are used herein interchangeably to refer to an antibody having a structure substantially similar to a native antibody structure or having heavy chains that contain an Fc region as defined herein.

“Antibody fragment” or “antigen binding fragment” refers to a portion of a full-length antibody which is capable of binding the same antigen as the full-length antibody. Examples of antibody fragments include, but are not limited to, Fv, Fab, Fab′, Fab′-SH, F(ab′)₂; diabodies; linear antibodies; single-chain antibody molecules (e.g., scFv); and multispecific antibodies formed from antibody fragments.

“Class” of an antibody refers to the type of constant domain or constant region possessed by its heavy chain. There are five major classes of antibodies: IgA, IgD, IgE, IgG, and IgM, and several of these are further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) (see, e.g., Kindt et al., Kuby Immunology, 6th ed., W.H. Freeman and Co., page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (see, e.g., Portolano et al., J. Immunol. 150:880-887 (1993); Clarkson et al., Nature 352:624-628 (1991)).

“Hypervariable region” or “HVR,” as used herein, refers to each of the regions of an antibody variable domain which are hypervariable in sequence and/or form structurally defined loops (“hypervariable loops”). Generally, native antibodies comprise four chains with six HVRs: three in the heavy chain variable domains, VH (H1, H2, H3), and three in the light chain variable domains, VL (L1, L2, L3). The HVRs generally comprise amino acid residues from the hypervariable loops and/or from the “complementarity determining regions” (CDRs). Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Unless otherwise indicated, HVR residues and other residues in the variable domain (e.g., FR residues) are numbered herein according to Kabat et al., Sequences of Proteins of Immunological Interest, 5th Ed. Public Health Service, National Institutes of Health, Bethesda, MD (1991).

“Complementarity determining region,” or “CDR,” as used herein, refers to the regions within the hypervariable regions of the variable domain which have the highest sequence variability and/or are involved in antigen recognition. Generally, native antibodies comprise four chains with six CDRs: three in the heavy chain variable domains, VH (H1, H2, H3), and three in the light chain variable domains, VL (L1, L2, L3). Exemplary CDRs (CDR-L1, CDR-L2 ,CDR-L3, CDR-H1, CDR-H2, and CDR-H3) occur at amino acid residues 24-34 of L1, 50-56 of L2, 89-97 of L3, 31-35 of H1, 50-65 of H2, and 95-102 of H3. (Kabat et al., supra). With the exception of CDR-H1 in VH, CDRs generally comprise the amino acid residues that form the hypervariable loops.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

“Native antibody” refers to a naturally occurring immunoglobulin molecule. For example, native IgG antibodies are heterotetrameric glycoproteins of about 150 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Monoclonal antibody” as used herein refers to an antibody obtained from a substantially homogeneous population of antibodies, i.e., the individual antibodies comprising the population are identical and/or bind the same epitope, except for possible variant antibodies (e.g., variant antibodies contain mutations that occur naturally or arise during production of a monoclonal antibody, and generally are present in minor amounts). In contrast to polyclonal antibody preparations, which typically include different antibodies directed against different determinants (epitopes), each monoclonal antibody of a monoclonal antibody preparation is directed against a single determinant on an antigen. Thus, the term “monoclonal” indicates the character of the antibody as being obtained from a substantially homogeneous population of antibodies and is not to be construed as requiring production of the antibody by any particular method. For example, the monoclonal antibodies to be used may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Chimeric antibody” refers to an antibody in which a portion of the heavy and/or light chain is derived from a particular source or species, while the remainder of the heavy and/or light chain is derived from a different source or species.

“Humanized antibody” refers to a chimeric antibody comprising amino acid sequences from non-human HVRs and amino acid sequences from human FRs. In certain embodiments, a humanized antibody will comprise substantially all of at least one, and typically two, variable domains, in which all or substantially all of the FTVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Isolated antibody,” as used herein, is intended to refer to an antibody that is substantially free of other antibodies having different antigenic specificities (e.g., an isolated antibody that specifically binds IFN-γ is substantially free of antibodies that specifically bind antigens other than IFN-γ). An isolated antibody that specifically binds IFN-γ may, however, have cross-reactivity to other antigens, such as IFN-γ molecules from other species. Moreover, an isolated antibody may be substantially free of other cellular material and/or chemicals.

“Human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

“Human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

“Recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germ line V_{H} and V_{L} sequences, may not naturally exist within the human antibody germline repertoire in vivo.

“Affinity” refers to the strength of the sum total of noncovalent interactions between a single binding site of a molecule (e.g., an antibody) and its binding partner (e.g., an antigen). “Binding affinity” refers to intrinsic binding affinity which reflects a 1:1 interaction between members of a binding pair (e.g., antibody and antigen). The affinity of a molecule X for its partner Y can generally be represented by the equilibrium dissociation constant (KD). Affinity can be measured by common methods known in the art, including those described herein. Specific illustrative and exemplary embodiments for measuring binding affinity are described in the following.

“Binds specifically” or “specific binding” refers to binding of an antibody to an antigen with an affinity value of no more than about 1×10⁻⁷ M.

“Treatment,” “treat” or “treating” refers to clinical intervention in an attempt to alter the natural course of a disorder in a subject being treated and can be performed either for prophylaxis or during the course of clinical pathology. Desired results of treatment can include, but are not limited to, preventing occurrence or recurrence of the disorder, alleviation of symptoms, diminishment of any direct or indirect pathological consequences of the disorder, preventing metastasis, decreasing the rate of progression, amelioration or palliation of a disease state, and remission or improved prognosis. For example, treatment of HBV infection can include administration of a therapeutically effective amount of pharmaceutical formulation comprising an IFN-γ antibody to a subject to prevent, delay development of, slow progression of, or eradicate an HBV infection.

“Pharmaceutical composition” or “composition” or “formulation” refers to a preparation in a form that allows the biological activity of the active ingredient(s) to be effective, and which contain no additional components which are toxic to the subjects to which the formulation is administered.

“Pharmaceutically acceptable carrier” refers to an ingredient in a pharmaceutical formulation, other than an active ingredient, which is nontoxic to the subject to whom it is administered. A pharmaceutically acceptable carrier includes, but is not limited to, a buffer, excipient, stabilizer, or preservative.

“Therapeutically effective amount,” as used herein, refers to the amount of an active ingredient or agent (e.g., a pharmaceutical composition) to achieve a desired therapeutic or prophylactic result, e.g., to treat or prevent a disease, disorder, or condition in a subject. In the case of a vitiligo subject, the therapeutically effective amount of the therapeutic agent is an amount that reduces, prevents, inhibits, and/or relieves to some extent one or more of the symptoms associated with the vitiligo. For therapeutic treatment of vitiligo, efficacy in vivo can, for example, be measured by assessing the duration, severity, and/or recurrence of symptoms, the response rate (RR), duration of response, and/or quality of life.

“Subject” refers to a mammal, including but not limited to, primates (e.g., humans and non-human primates such as monkeys), rodents (e.g., mice and rats), rabbits, and domesticated animals (e.g., cows, sheep, cats, dogs, and horses).

“Subject in need” as referred to herein includes patients with a vitiligo.

### Methods for Treatment of Vitiligo Using IFN-γ Antibodies

The present disclosure provides methods for treating vitiligo, comprising administering to a subject in need thereof a composition comprising a therapeutically effective amount of an IFN-γ antibody and a pharmaceutically acceptable carrier. Various IFN-γ antibody compositions, and modes of administration useful in the methods of treatment are described in greater detail below and exemplified in the Examples. Additionally, methods of treatment further comprising administering an additional therapeutic agent are further described below and exemplified in the Examples.

IFN-γ is a dimerized soluble cytokine that is a member of the type II class of interferons. IFN-γ is an important immunostimulatory and immunomodulatory molecule that functions as activator of macrophages and inducer of Class II major histocompatibility complex (MHC) molecule expression. IFN-γ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops. Aberrant expression of IFN-γ has been associated with a number of autoinflammatory and autoimmune diseases. IFN-γ importance in the immune system is thought to arise at least in part from an ability to inhibit viral replication. The amino acid and nucleotide sequences and annotation of the human, primate, and other mammalian versions of IFN-γ are publicly available. See e.g., full amino acid sequence of human IFN-γ at UniProt entry number P01579. The amino acid sequence of human IFN-γ is disclosed as SEQ ID NO: 166 of the accompanying Sequence Listing.

Generally, the IFN-γ antibodies useful in the methods for treatment of vitiligo have the functional characteristic of decreasing, inhibiting, and/or blocking (partially or fully) IFN-γ signaling activity. In some embodiments, the IFN-γ antibodies useful in the methods of the present disclosure are capable of modulating a biological function of IFN-γ, neutralizing IFN-γ, or decreasing IFN-γ binding to its receptor. In some embodiments, the IFN-γ antibody may decrease, inhibit, and/or block the signaling activity of pro-human IFN-γ, mature-human IFN-γ, or truncated-human IFN-γ, and/or the ability of pro-human IFN-γ, mature-human IFN-γ, or truncated-human IFN-γ to bind to its receptor; and thereby reduce one or more of IFN-γ-dependent cytokine production, IFN-γ-dependent T cell dysfunction, IFN-γ-dependent immune tolerance,, and/or IFN-γ-dependent inflammation.

Exemplary IFN-γ antibodies useful in the methods of the present disclosure can include, for example, IFN-γ antagonists or inhibitors that modulate at least one biological function or activity of IFN-γ. Biological functions or activities of IFN-γ include, for example, binding the IFN-γ receptor (IFN-γ-R), modulating (e.g., enhancing) major histocompatibility complex (MHC) class II expression on a cell surface, modulating (e.g., reducing or inhibiting) cell proliferation, and/or modulating an immune response.

In some embodiments the IFN-γ antibodies useful in the methods of the present disclosure completely or partially inhibit IFN-γ signaling activity. In some embodiments, the IFN-γ antibodies completely or partially inhibit IFN-γ signaling activity by partially or completely blocking the binding of IFN-γ to the IFN-γ receptor. It is contemplated, however, that the IFN-γ antibodies can also completely or partially inhibit IFN-γ signaling by mechanisms that do not involve direct inhibition of IFN-γ binding to the IFN-γ receptor.

In some embodiments, the IFN-γ antibodies useful in the methods and compositions of the present disclosure can be described in terms on the amino acid and encoding nucleotide sequences of the various well-known immunoglobulin features (e.g., CDRs, HVRs, FRs, V_{H}, and V_{L} domains). Table 1 below provides a summary description of sequences and sequence identifiers for exemplary IFN-γ antibodies useful in the methods of the present disclosure, including the IFN-γ antibodies described elsewhere herein as “2A6,” “2B6,” “2A6_Q,” (or “2A6Q”) and “2A6_A” (or “2A6A”). The sequences are included in the accompanying Sequence Listing.

TABLE 1
IFN-γ antibody sequences

| | | SEQ |
|---|---|---|
| | | ID |
| Description | Sequence | NO: |
| 2A6-VH | EVQLVESGGGLVKPGDSLTVSCVASGFTFSNYFIH | 109 |
| | WVRQAPGKGLEWVATISGRTKYMFYSDSLRGRFT | |
| | VSRDNANNSVYLHMSSLRGEDTALYYCVRGYDHS | |
| | DSNSAADLLHWGRGTLVTVSS | |
| 2A6-CDR-H1-IMGT | GFTFSNYF | 120 |
| 2A6-CDR-H2-IMGT | ISGRTKYM | 121 |
| 2A6-CDR-H3-IMGT | VRGYDHSDSNSAADLLH | 122 |
| 2A6-CDR-H1-Kabat | GFTFSNYFIH | 144 |
| 2A6-CDR-H2-Kabat | TISGRTKYMFYSDSLRG | 145 |
| 2A6-CDR-H3-Kabat | GYDHSDSNSAADLLH | 146 |
| 2B6-VH | EVQLVESGGGLVKPGGSLRLSCAASGFPFSRYSMH | 110 |
| | WVRQAPGKGLEWVSSMTSRTNHKYYADSLKGRF | |
| | LISRDNDRDSLYLEMNSLGVEDTAIYFCARGYDTS | |
| | GSDSGVDFQYWGQGTLVTVSS | |
| 2B6-CDR-H1-IMGT | GFPFSRYS | 123 |
| 2B6-CDR-H2-IMGT | MTSRTNHK | 124 |
| 2B6-CDR-H3-IMGT | ARGYDTSGSDSGVDFQY | 125 |
| 2B6-CDR-H1-Kabat | GFPFSRYSMH | 147 |
| 2B6-CDR-H2-Kabat | SMTSRTNHKYYADSLKG | 148 |
| 2B6-CDR-H3-Kabat | GYDTSGSDSGVDFQY | 149 |
| 2A6-VL | NFMLTQPLSVSGYPGKTIVISCVRSSGSVASHYVQW | 113 |
| | FQQRPGSAPAMVIYEDSHRPSGIPDRESGSVDASSNS | |
| | ASLTISGLKTEDEADYFCQSYYGNNQVLFGGGTKL | |
| | TVL | |
| 2A6-CDR-L1-IMGT | SGSVASHY | 132 |
| 2A6-CDR-L2-IMGT | EDS | 133 |
| 2A6-CDR-L3-IMGT | QSYYGNNQVL | 134 |
| 2A6-CDR-L1-Kabat | VRSSGSVASHYVQ | 156 |
| 2A6-CDR-L2-Kabat | EDSHRPS | 157 |
| 2A6-CDR-L3-Kabat | QSYYGNNQVL | 134 |
| 2B6-VL | NFMLTQPHSVSESPGKTITISCTRGRGYIASYYVQW | 114 |
| | YQQRPGGVPKIVVFEDTQRPSGVPDRISGSIDTSTNS | |
| | ASLTISGLQTEDEADYYCQSYDDANHVIFGGGTKL | |
| | TVL | |
| 2B6-CDR-L1-IMGT | RGYIASYY | 135 |
| 2B6-CDR-L2-IMGT | EDT | 136 |
| 2B6-CDR-L3-IMGT | QSYDDANHVI | 137 |
| 2B6-CDR-L1-Kabat | TRGRGYIASYYVQ | 158 |
| 2B6-CDR-L2-Kabat | EDTQRPS | 159 |
| 2B6-CDR-L3-Kabat | QSYDDANHVI | 137 |
| 2A6_A-VH | EVQLVESGGGLVKPGDSLTVSCVASGFTFSNYFIH | 164 |
| | WVRQAPGKGLEWVATISGRTKYMFYSDSLRGRFT | |
| | VSRDNAANSVYLHMSSLRGEDTALYYCVRGYDHS | |
| | DSNSAADLLHWGRGTLVTVSS | |
| 2A6_Q-VH | EVOLVESGGGLVKPGDSLTVSCVASGFTFSNYFIH | 165 |
| | WVRQAPGKGLEWVATISGRTKYMFYSDSLRGRFT | |
| | VSRDNAQNSVYLHMSSLRGEDTALYYCVRGYDHS | |
| | DSNSAADLLHWGRGTLVTVSS | |
| 2A6-Heavy Chain | EVQLVESGGGLVKPGDSLTVSCVASGFTFSNYFIHW | 183 |
| | VRQAPGKGLEWVATISGRTKYMFYSDSLRGRFTVS | |
| | RDNANNSVYLHMSSLRGEDTALYYCVRGYDHSDS | |
| | NSAADLLHWGRGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNHKP | |
| | SNTKVDKKVEPKSCDKTHTCPPCPAPELLGGPSVFL | |
| | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSREEMTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |
| 2A6-Light Chain | NFMLTQPLSVSGYPGKTIVISCVRSSGSVASHYVQW | 184 |
| | FQQRPGSAPAMVIYEDSHRPSGIPDRESGSVDASSNS | |
| | ASLTISGLKTEDEADYFCQSYYGNNQVLFGGGTKL | |
| | TVLGQPKANPTVTLFPPSSEELQANKATLVCLISDFY | |
| | PGAVTVAWKADGSPVKAGVETTKPSKQSNNKYAA | |
| | SSYLSLTPEQWKSHRSYSCQVTHEGSTVEKTVAPTE | |
| | CS | |
| 2B6-Heavy Chain | EVQLVESGGGLVKPGGSLRLSCAASGFPFSRYSMH | 185 |
| | WVRQAPGKGLEWVSSMTSRTNHKYYADSLKGRFL | |
| | ISRDNDRDSLYLEMNSLGVEDTAIYFCARGYDTSGS | |
| | DSGVDFQYWGQGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNHKP | |
| | SNTKVDKKVEPKSCDKTHTCPPCPAPELLGGPSVFL | |
| | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSREEMTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |
| 2B6-Light Chain | NFMLTQPHSVSESPGKTITISCTRGRGYIASYYVQW | 186 |
| | YQQRPGGVPKIVVFEDTQRPSGVPDRISGSIDTSTNS | |
| | ASLTISGLQTEDEADYYCQSYDDANHVIFGGGTKLT | |
| | VLGQPKANPTVTLFPPSSEELQANKATLVCLISDFYP | |
| | GAVTVAWKADGSPVKAGVETTKPSKQSNNKYAAS | |
| | SYLSLTPEQWKSHRSYSCQVTHEGSTVEKTVAPTEC | |
| | S | |
| 2A6_A-Heavy Chain | EVQLVESGGGLVKPGDSLTVSCVASGFTFSNYFIHW | 187 |
| | VRQAPGKGLEWVATISGRTKYMFYSDSLRGRFTVS | |
| | RDNAANSVYLHMSSLRGEDTALYYCVRGYDHSDS | |
| | NSAADLLHWGRGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNHKP | |
| | SNTKVDKKVEPKSCDKTHTCPPCPAPELLGGPSVFL | |
| | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSREEMTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |
| 2A6_Q-Heavy Chain | EVQLVESGGGLVKPGDSLTVSCVASGFTFSNYFIHW | 188 |
| | VRQAPGKGLEWVATISGRTKYMFYSDSLRGRFTVS | |
| | RDNAQNSVYLHMSSLRGEDTALYYCVRGYDHSDS | |
| | NSAADLLHWGRGTLVTVSSASTKGPSVFPLAPSSKS | |
| | TSGGTAALGCLVKDYFPEPVTVSWNSGALTSGVHT | |
| | FPAVLQSSGLYSLSSVVTVPSSSLGTQTYICNVNHKP | |
| | SNTKVDKKVEPKSCDKTHTCPPCPAPELLGGPSVFL | |
| | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSREEMTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |

Additional, IFN-γ antibodies useful in the methods and compositions of the present disclosure include any antibody known in the art to specifically bind IFN-γ and neutralize, inhibit, decrease, and/or otherwise block the IFN-γ signaling activity. Exemplary antibodies known in the art include the following IFN-γ antibodies: AMG-811 (described in e.g., US Pat. No. 7,335,743 B2), NI-0501 (described in e.g., U.S. Pat. No. 7,700,098 B2), HuZAF (described in e.g., U.S. Pat. No. 6,329,511 B1). The disclosure of each of U.S. Pat. Nos. 7,335,743 B2, 7,700,098 B2, and 6,329,511 B1, are hereby incorporated herein in its entirety by reference.

Generally, the IFN-γ antibodies useful in the methods of treatment of the present disclosure exhibit high-affinity binding to IFN-γ. In some embodiments, the anti-IFN-γ antibodies provided herein have an equilibrium dissociation constant (K_{D}) for binding to IFN-γ of <100 nM, <10 nM, <1 nM, <0.1 nM, <0.01 nM, or <0.001 nM (e.g., 10⁻⁸ M or less, from 10⁻⁸ M to 10⁻¹³ M, e.g., from 10⁻⁹ M to 10⁻¹³ M).

Further, the anti-IFN-γ antibodies useful in the methods disclosed herein include antibodies capable of high-affinity binding to human IFN-γ, cynomolgus monkey IFN-γ, and in some embodiments, high-affinity binding to both human IFN-γ and cynomolgus IFN-γ. More specifically, in some embodiments, the IFN-γ antibodies useful in the methods of the present disclosure bind to human IFN-γ with a binding affinity of 1×10⁻⁸ M or less, 1×10⁻⁹ M or less, 1×10⁻¹⁰ M or less, or 1×10⁻¹¹ M or less. In some embodiments, the anti-IFN-γ antibodies of the present disclosure bind to cynomolgus IFN-γ with a binding affinity of 1×10⁻⁸ M or less, 1×10⁻⁹ M or less, 1×10⁻¹⁰ M or less, or 1×10⁻¹¹ M or less. In some embodiments, the anti-IFN-γ antibodies of the present disclosure bind to both human IFN-γ and cynomolgus IFN-γ with a binding affinity of 1×10⁻⁸ M or less, 1×10⁻⁹ M or less, 1×10⁻¹⁰ M or less, or 1×10⁻¹¹ M or less.

Generally, the binding affinity of IFN-γ antibodies can be determined using any of a variety of assays and expressed in terms of a variety of quantitative values. Specific IFN-γ binding assays useful in determining affinity of the antibodies are disclosed in the Examples herein. Additionally, antigen binding assays are known in the art and can be used herein including without limitation any direct or competitive binding assays using techniques such as western blots, radioimmunoassays, enzyme-linked immunoabsorbent assay (ELISA), “sandwich” immunoassays, surface plasmon resonance-based assay (such as the BIAcore assay as described in WO2005/012359), immunoprecipitation assays, fluorescent immunoassays, and protein A immunoassays.

In some embodiments, the anti-IFN-γ antibodies useful in the methods of the present disclosure decrease, inhibit, and/or fully-block IFN-γ binding to its cognate receptor, IFN-γ-R, and thereby modulate immune regulation and/or immune signaling mediated by IFN-γ. The ability of the IFN-γ antibodies to inhibit these immune regulatory and immune signaling pathways mediated by IFN-γ binding can be assayed in vitro using known cell-based assays including the various cell-based assays described in the Examples of the present disclosure.

Accordingly, in some embodiments, the IFN-γ antibodies useful in the methods of the present disclosure are characterized by an ability to enhance a measurable immune response in human PBMCs stimulated by staphylococcal enterotoxin B (SEB) by at least 1.2-fold, 1.3-fold, 1.4-fold, 1.5-fold, 1.6-fold, 1.7-fold, 1.8-fold, at least 1.9-fold, at least 2-fold, at least 2.1-fold, or at least 2.20-fold. For example, in some embodiments, the IFN-γ antibody increases IL-2 production and/or cell proliferation in SEB-stimulated human PBMCs by at least,2-fold, 1.3-fold, 1.4-fold, 1.5-fold, 1.6-fold, 1.7-fold, 1.8-fold, at least 1.9-fold, at least 2-fold, at least 2.1-fold, or at least 2.20-fold. Additional, functional characteristics of exemplary IFN-γ antibodies useful in the methods of the present disclosure are described further below and in the Examples.

As described above, it is contemplated that the IFN-γ antibodies useful in the methods for treating vitiligo of the present disclosure can include any immunoglobulin comprising one or more polypeptide chains that specifically binds to or is immunologically reactive with IFN-γ. Accordingly, IFN-γ antibodies useful in the methods of the present disclosure an include native antibodies, whole antibodies, monoclonal antibodies, polyclonal antibodies, chimeric antibodies, humanized antibodies, human antibodies, multispecific (or heteroconjugate) antibodies (e.g., bispecific antibodies), monovalent antibodies, multivalent antibodies, antigen-binding antibody fragments (e.g., Fab′, F(ab′)₂, Fab, Fv, rIgG, and scFv fragments), antibody fusions, and synthetic antibodies (or antibody mimetics).

In some embodiments of the present disclosure the IFN-γ antibody is a glycoprotein comprising at least two heavy (H) chains and two light (L) chains inter-connected by disulfide bonds, or an antigen binding portion thereof. Each heavy chain comprises a heavy chain variable region (abbreviated herein as V_{H}) and a heavy chain constant region. The heavy chain constant region is comprised of three domains, CH1, CH2, and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

In some embodiments according to the present disclosure, the IFN-γ antibody may be an immunoglobulin molecule, an Fv, a disulfide linked Fv, a monoclonal antibody, an scFv, a chimeric antibody, a single domain antibody, a CDR-grafted antibody, a diabody, a humanized antibody, a multispecific antibody, an Fab, a dual specific antibody, an Fab' fragment, a bispecific antibody, an F(ab′)₂ fragment, a bivalent fragment comprising two Fab fragments linked by a disulfide bridge at the hinge region; a Fd fragment consisting of the V_{H} and CHI domains; a Fv fragment consisting of the V_{L} and V_{H} domains of a single arm of an antibody, a dAb fragment, an isolated complementary determining region (CDR), or a single chain antibody. Particularly, the IFN-γ antibody may be a human protein or a humanized binding protein.

In some embodiments of the present disclosure the IFN-γ antibody is an “antigen-binding fragment” such as a diabody, a Fab, a Fab′, a F(ab′)₂, an Fv fragment, a disulfide stabilized Fv fragment (dsFv), a (dsFv)₂, a bispecific dsFv (dsFv-dsFv'), a disulfide stabilized diabody (ds diabody), a single-chain antibody molecule (scFv), an scFv dimer (bivalent diabody), a multispecific antibody formed from a portion of an antibody comprising one or more CDRs, or any other antibody fragment that binds to an antigen but does not comprise a complete antibody structure. In such embodiments, the antigen-binding fragment is capable of binding to the same IFN-γ antigen to which the parent antibody or a parent antibody fragment (e.g., a parent scFv) binds. In certain embodiments, the antigen-binding fragment may comprise one or more CDRs from a particular human antibody grafted to a framework region from one or more different human antibodies.

Among the above-described antigen-binding fragments, a Fab, which is a structure having the light chain and heavy chain variable regions, the light chain constant region, and the heavy chain first constant region (CH1), has one antigen binding site. A Fab′ differs from the Fab in that the Fab′ has a hinge region including at least one cysteine residue at the C-terminal of the heavy chain CH1 domain. A F(ab′)₂ is produced when cysteine residues at the hinge region of Fab′ are joined by a disulfide bond.

An Fv is a minimal antibody fragment, having only heavy chain variable regions and light chain variable regions, and a recombinant technique for producing the Fv fragment is well known in the art. “Single-chain Fv antibody” or “scFv” refers to an engineered antibody consisting of a light chain variable region and a heavy chain variable region connected to one another directly or via a peptide linker sequence. A two-chain Fv may have a structure in which heavy chain variable regions are linked to light chain variable regions by a non-covalent bond, and a single-chain Fv may generally form a dimer structure as in the two-chain Fv, wherein heavy chain variable regions are covalently bound to light chain variable regions via a peptide linker or the heavy and light chain variable regions are directly linked to each other at the C-terminals thereof. The linker may be a peptide linker including 1 to 100 or 2 to 50 any amino acids, and proper sequences thereof have been known in the art.

The antigen-binding fragment may be obtained using a protease (for example, a whole antibody can be digested with papain to obtain Fab fragments, can be digested with pepsin to obtain F(ab′)₂ fragments), or may be prepared by a genetic recombinant technique.

In some embodiments of the methods of the present disclosure, the IFN-γ antibody can be a multispecific antibody, e.g., a bispecific antibody. In some embodiments, the multispecific antibody is a monoclonal antibody having at least two different binding sites, each with a binding specificity for a different antigen, at least one of which specifically binds IFN-γ. In some embodiments, the multispecific antibody is a bispecific antibody comprising a specificity for IFN-γ and a specificity for another antigen that mediates immune regulation, and/or immune signaling. In some embodiments of the bispecific antibody, the other specificity is for an antigen that is an immune checkpoint molecule selected from PD1, PD-L1, CTLA-4, TIGIT, LAG3, PVRIG, KIR, TIM-3, CRTAM, BTLA, CD244, CD160, LIGHT, GITR, 4-1BB, OX40, CD27, TMIGD2, ICOS, CD40, CD47, SIRPa, NKG2D, NKG2A, TNFRSF25, CD33, CEA, Epcam, GPC3, CD200, CD200R, CD73, CD83, CD39, TRAIL, CD226, and VISTA. In some embodiment the anti-IFN-γ bispecific antibody, the other antigen for which the antibody has specificity is selected from PD1, PD-L1, and CTLA-4.

Techniques for making multispecific antibodies include, but are not limited to, recombinant co-expression of two immunoglobulin heavy chain-light chain pairs having different specificities (see e.g., Milstein and Cuello, Nature 305:537 (1983), WO 93/08829, and Traunecker et al., EMBOJ. 10:3655 (1991)). “Knob-in-hole” engineering can also be used to generate bispecific antibodies useful with the anti-IFN-γ antibodies of the present disclosure. Techniques for knob-in-hole engineering are known in the art and described in e.g., U.S. Pat. No. 5,731,168.

In some embodiments, the IFN-γ antibody useful in the methods of the present disclosure can be altered to increase or decrease the extent to which the antibody is glycosylated. Addition or deletion of glycosylation sites to an antibody can be carried out by altering the amino acid sequence such that one or more glycosylation sites is created or removed. For instance, the present disclosure provides exemplary IFN-γ antibodies 2A6_A and 2A6_Q that have their sequences modified to remove an N-linked glycosylation site at position 76 of the V_{H} region.

It also is contemplated that carbohydrates attached to the Fc region can be altered. Typically, native antibodies produced by mammalian cells comprise a branched, biantennary oligosaccharide attached by an N-linkage to the asparagine at about position 297 (“N297”) of the CH2 domain of the Fc region (see, e.g., Wright et al. TIBTECH 15:26-32 (1997)). The oligosaccharide may include various carbohydrates, such as mannose, N-acetyl glucosamine (GlcNAc), galactose, and sialic acid, as well as, a fucose attached to a GlcNAc in the “stem” of the bi-antennary oligosaccharide structure. In some embodiments, the modifications of the oligosaccharide of an Fc region of an antibody can create a variant with certain improved properties. In some embodiments, the IFN-γ antibody of the present disclosure can be a variant of a parent antibody, wherein the variant comprises a carbohydrate structure that lacks fucose attached (directly or indirectly) to an Fc region. For example, the amount of fucose in such antibody may be from about 1% to about 80%, from about 1% to about 65%, from about 5% to about 65%, or from about 20% to about 40%. The amount of fucose can be determined by calculating the average amount of fucose within the sugar chain at N297, relative to the sum of all glyco-structures attached to Asn 297 (e.g., complex, hybrid and high mannose structures) as measured by MALDI-TOF mass spectrometry (see e.g., WO 2008/077546). N297 refers to the asparagine residue located at about position 297 in the Fc region (Eu numbering of Fc region residues); however, N297 may also be located about ±3 amino acids upstream or downstream of position 297, i.e., between positions 294 and 300, due to minor sequence variations in antibodies.

In some embodiments, the fucosylation variants can have improved ADCC function. See, e.g., US Patent Publication Nos. US 2003/0157108, or US 2004/0093621. Examples of “defucosylated” or “fucose-deficient” antibodies and associated methods for preparing them are disclosed in e.g., US2003/0157108; US2003/0115614; US2002/0164328; US2004/0093621; US2004/0132140; US2004/0110704; US2004/0110282; US2004/0109865; WO2000/61739; WO2001/29246; WO2003/085119; WO2003/084570; WO2005/035586; WO2005/035778; WO2005/053742; WO2002/031140; Okazaki et al. J. Mol. Biol. 336:1239-1249 (2004); Yamane-Ohnuki et al. Biotech. Bioeng. 87:614 (2004).

Cell lines useful for producing defucosylated antibodies include Led 3 CHO cells deficient in protein fucosylation (see e.g., Ripka et al. Arch. Biochem. Biophys. 249:533-545 (1986); US2003/0157108, and WO2004/056312), and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

In some embodiments, IFN-γ antibodies useful in the methods of the present disclosure can comprise one or more amino acid modifications in the Fc region (i.e., an Fc region variant). The Fc region variant may comprise a human Fc region sequence (e.g., a human IgG1, IgG2, IgG3, or IgG4 Fc region) comprising an amino acid substitution at one or more amino acid residue positions. A wide range of Fc region variants known in the art that are useful with the anti-IFN-γ antibodies of the present disclosure are described below.

In some embodiments, it is contemplated that the IFN-γ antibody is an Fc region variant which has altered effector function. In some embodiments, the antibody with altered effector function possesses some (but not all of) the effector functions, decreased effector function, or none of the effector functions (e.g., effectorless) of the parent antibody. Effectorless Fc region variants are more desirable for certain applications where effector function (such as ADCC) is unnecessary or deleterious, and/or in vivo half-life of the antibody is important. Fc region variant antibodies with reduced effector function, or which are effectorless, can include an amino acid substitution at one or more of the following Fc region positions: 238, 265, 269, 270, 297, 327 and 329. (See, e.g., U.S. Pat. No. 6,737,056). Such Fc region variants can include amino acid substitutions at two or more of positions 265, 269, 270, 297 and 327. Such Fc region variants can also include substitutions of both residues 265 and 297 to alanine (see e.g., US Pat. No. 7,332,581).

Accordingly, in one embodiment the present disclosure provides a method for treating vitiligo, comprising administering to a subject in need thereof a composition comprising a therapeutically effective amount of an IFN-γ antibody and a pharmaceutically acceptable carrier.

Thus, the exemplary cell-based model studies support a mechanism whereby an IFN-γ antibody can neutralize cell-based pathways that are associated with pathogenesis of vitiligo. In other words, the present invention suggests that an IFN-γ antibody can be used to provide an improved method for the treatment of vitiligo.

### Modes for Administering IFN-γ Antibody Compositions

Administration to a subject in need of a composition or formulation comprising an IFN-y antibody in accordance with the methods of treatment provides a therapeutic effect that protects the subject from and/or treats vitiligo.

In some embodiments of the methods of treatment of the present disclosure, the IFN-γ antibody composition or formulation comprising an IFN-γ antibody is administered to a subject by any mode of administration that delivers the agent systemically, or to a desired target tissue. Systemic administration generally refers to any mode of administration of the antibody into a subject at a site other than directly into the desired target site, tissue, or organ, such that the antibody or formulation thereof enters the subject's circulatory system and, thus, is subject to metabolism and other like processes.

Accordingly, modes of administration useful in the methods of treatment of vitiligo of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

In an embodiment according to the methods of the present disclosure, the IFN-γ antibody may be administered to the subject in need thereof by at least one route selected from the group consisting of parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal. In one embodiment, the IFN-γ antibody may be administered to the subject in need intravenously.

In some embodiments, a formulation of the IFN-γ antibody is formulated such that the antibody is protected from inactivation in the gut. Accordingly, the method of treatments can comprise oral administration of the formulation.

In some embodiments, the present disclosure provides uses of compositions or formulations comprising an IFN-γ antibody as a medicament for the treatment of vitiligo. Additionally, in some embodiments, the present disclosure also provides for the use of a composition or a formulation comprising an IFN-γ antibody in the manufacture or preparation of a medicament for the treatment of vitiligo. In a further embodiment, the medicament is for use in a method for treating vitiligo comprising administering to a subject in need thereof an effective amount of the medicament. In certain embodiments, the medicament further comprises an effective amount of at least one additional therapeutic agent, or treatment.

In a further embodiment, the medicament is for use in treating vitiligo in a subject comprising administering to the subject an amount effective of the medicament to treat the vitiligo.

For the treatment of vitiligo, the appropriate dosage of the IFN-γ antibody contained in the compositions and formulations of the present disclosure (when used alone or in combination with one or more other additional therapeutic agents) will depend on factors including the severity and course of the disease, whether the antibody is administered for preventive or therapeutic purposes, the previous therapy administered to the patient, the patient's clinical history and response to the antibody, and the discretion of the attending physician.

Generally, a treatment regimen useful in the methods of the present disclosure can be decided by the medical personnel of the subject in need. The IFN-γ antibodies of the present disclosure when included in the compositions and formulations described herein, can be suitably administered to the patient at one time, or over a series of treatments. Various dosing schedules including but not limited to single or multiple administrations over various time-points, bolus administration, and pulse infusion are contemplated herein.

In some embodiments of the methods, the composition comprising an IFN-γ antibody may be administered to the subject in need more than once a day, at least once a day, at least once a week, or at least once a month.

Depending on the type and severity of the vitiligo, about 1 μg/kg to 15 mg/kg of IFN-γ antibody in a formulation of the present disclosure is an initial candidate dosage for administration to a human subject, whether, for example, by one or more separate administrations, or by continuous infusion. Generally, the administered dosage of the antibody would be in the range from about 0.05 mg/kg to about 10 mg/kg. In some embodiments, one or more doses of about 0.5 mg/kg, 2.0 mg/kg, 4.0 mg/kg or 10 mg/kg (or any combination thereof) may be administered to a patient.

Dosage administration can be maintained over several days or longer, depending on the condition of the subject, for example, administration can continue until the vitiligo is sufficiently treated, as determined by methods known in the art. In some embodiments, an initial higher loading dose may be administered, followed by one or more lower doses. However, other dosage regimens may be useful. The progress of the therapeutic effect of dosage administration can be monitored by conventional techniques and assays.

Accordingly, in some embodiments of the methods of the present disclosure, the administration of the IFN-γ antibody comprises a daily dosage from about 1 mg/kg to about 100 mg/kg. In some embodiments, the dosage of IFN-γ antibody comprises a daily dosage of at least about 1 mg/kg, at least about 5 mg/kg, at least about 10 mg/kg, at least about 20 mg/kg, or at least about 30 mg/kg.

### Combination Treatment Methods With Other Therapeutic Agents

In an embodiment according to the present invention, the method may further comprise the step of administering at least one additional therapeutic agent. For example, the additional therapeutic agent may be administered to the subject in need thereof in combination with the IFN-y antibody composition—e.g., administered at the same time as the IFN-γ antibody composition; before administration of the IFN-γ antibody composition; or after administration of the IFN-γ antibody composition. In some embodiments, the additional therapeutic agent may comprise an additional treatment for vitiligo or a treatment for a disease or condition associated with vitiligo. It is contemplated that in the combination treatment method, by administering an IFN-γ antibody composition in combination with a therapeutic agent the efficacy of the therapeutic agent may be improved. Without being bound by theory, it is believed that after administering a therapeutically effective amount of the IFN-γ antibody composition and an optional additional therapeutic agent, an immune response related to vitiligo may be reduced or ameliorated in the subject in need, even to a level when it is undetectable. Additionally, the reduced or ameliorated immune response may comprise reduction of antibodies or cytokines that further modulate the activity of the immune system.

In one embodiment, the method comprising administering at least one additional therapeutic agent is carried out wherein the additional therapeutic agent is an IFN-γ antagonist. As described elsewhere herein, the IFN-γ antagonist useful in the combination treatment method can include IFN-γ antagonist which decrease the level of at least one biological activity of IFN-γ for therapeutic agent for treatment of subjects already have vitiligo. In some embodiments, the therapeutic agent is selected from a second IFN-γ neutralizing antibody, IFN-γ receptor antagonist and small molecule inhibitor to IFN-γ or IFN-γ receptor.

In other embodiments according to the present invention, the additional therapeutic agent may be selected from the group consisting of: a therapeutic agent; an imaging agent; a cytotoxic agent; an angiogenesis inhibitor; a kinase inhibitor; a co-stimulation molecule blocker; an adhesion molecule blockers; an anti-cytokine antibody or functional fragment thereof; methotrexate; cyclosporin; rapamycin; FK506; a detectable label or reporter; a TNF antagonist; an anti-rheumatic; a muscle relaxant; a narcotic; a non-steroid anti-inflammatory drug (NSAID); an analgesic; an anesthetic; a sedative; a local anesthetic; a neuromuscular blocker; an antimicrobial; an antipsoriatic; a corticosteroid; an anabolic steroid; an erythropoietin; an immunization; an immunoglobulin; an immunosuppressive; a growth hormone; a hormone replacement drug; a radiopharmaceutical; an antidepressant; an antipsychotic; a stimulant; an asthma medication; a beta agonist; an inhaled steroid; an epinephrine or analog thereof; a cytokine; and a cytokine antagonist; an immunomodulatory agent.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: IFN-γ Antibodies

### A. Recombinant IFN-γ Antibody Production

FreeStyle™ 293-F cells (Thermo Scientific, R79007) were cultured in a 250-mL flask containing FreeStyle™ 293 expression medium (Gibco, 12338018) under standard conditions with the concentration of 1×10⁶ cells. Transient transfection of the exponentially growing FreeStyle™ 293-F cells (1.5-2×10⁶ cells) were performed by linear polyethylenimine (PEI) with an average molecular weight of 25 kDa (Polysciences, Warrington, Pa) as a transfection reagent and a total of 88 μg of the plasmid DNA. After transfection, the cells were cultured for 3 days, and the culture medium was harvested. The culture medium was centrifuged for 10 min at 3000 rpm to remove the FreeStyle™ 293-F cell debris and afterward, the resultant supernatant was collected and filtered through a 0.45 um filter.

### B. Recombinant Antibody Purification

The resultant supernatants as obtained above were subsequently purified with Protein A Sepharose Fast Flow beads (GE Healthcare, 17-1279-01) so as to obtain the recombinant antibodies. In brief, 80 mL of the supernatants were added with 80 μL Protein A Sepharose Fast Flow beads and aliquoted evenly into two 50-mL tubes which were incubated for 24 hours at 4° C. under rotation. Then, the tubes were centrifuged at 3000 rpm for 10 min, and afterward, the resultant supernatants were removed, and the beads were equilibrated with PBS. The equilibrated beads were eluted with 0.1 M glycine (pH 3.0), and the eluates were collected in tubes containing 1 M Tris (pH 8.0) and dialyzed against PBS buffer, so as to obtain the monoclonal antibodies including 2A6, 2B6, 2A6A, 2A6Q, and BA anti-INF-γ mAbs, respectively.

For clarity, the CDRs on the V_{H} chain and V_{L} chain of each of the monoclonal antibodies are summarized in Table 2. CDRs were identified based on Kabat (Wu, T. T. and Kabat, E. A., 1970 J. Exp. Med. 132:211-250) and IMGT systems (Lefranc M.-P. et al., 1999 Nucleic Acids Research, 27, 209-212) by sequence annotation and by internet-based sequence analysis as described at e.g., www.imgt.org/IMGT_vquest/share/textes/index.html, and www.ncbi.nlm.nih.gov/igblast. In Table 2 below, CDRs following “K:” are based on Kabat system, and CDRs following “I:” are based on IMGT system.

TABLE 2

| mAb | V_{H} CDR1 | V_{H} CDR2 | V_{H} CDR3 |
|---|---|---|---|
| 2A6 | I: GFTFSNYF | I: ISGRTKYM | I: |
| | (SEQ ID No. 120) | (SEQ ID No. 121) | VRGYDHSDSNSAADLL |
| | | | H |
| | | | (SEQ ID No. 122) |
| | K: GFTFSNYFIH | K: | K: |
| | (SEQ ID No. 144) | TISGRTKYMFYSDSLR | GYDHSDSNSAADLLH |
| | | G (SEQ ID No. 145) | (SEQ ID No. 146) |
| 2B6 | I: GFPFSRYS | I: MTSRTNHK | I: |
| | (SEQ ID No. 123) | (SEQ ID No. 124) | ARGYDTSGSDSGVDFQ |
| | | | Y |
| | | | (SEQ ID No. 125) |
| | K: GFPFSRYSMH | K: | K: |
| | (SEQ ID No. 147) | SMTSRTNHKYYADSL | GYDTSGSDSGVDFQY |
| | | KG (SEQ ID No. 148) | (SEQ ID No. 149) |
| 2A6A | I: GFTFSNYF | I: ISGRTKYM | I: |
| | (SEQ ID No. 120) | (SEQ ID No. 121) | VRGYDHSDSNSAADLL |
| | | | H |
| | | | (SEQ ID No. 122) |
| | K: GFTFSNYFIH | K: | K: |
| | (SEQ ID No. 144) | TISGRTKYMFYSDSLR | GYDHSDSNSAADLLH |
| | | G | (SEQ ID No. 146) |
| | | (SEQ ID No. 145) | |
| 2A6Q | I: GFTFSNYF | I: ISGRTKYM | I: |
| | (SEQ ID No. 120) | (SEQ ID No. 121) | VRGYDHSDSNSAADLL |
| | | | H |
| | | | (SEQ ID No. 122) |
| | K: GFTFSNYFIH | K: | K: |
| | (SEQ ID No. 144) | TISGRTKYMFYSDSLR | GYDHSDSNSAADLLH |
| | | G | (SEQ ID No. 146) |
| | | (SEQ ID No. 145) | |
| BA | K: GFTFSNYFIH | K: | K: |
| | (SEQ ID No. 144) | TISGRTKYMFYSDSLR | GYDHSDSNSAADLLH |
| | | G | (SEQ ID No. 146) |
| | | (SEQ ID No. 145) | |
| | K: GFPFSRYSMH | K: | K: |
| | (SEQ ID No. 147) | SMTSRTNHKYYADSL | GYDTSGSDSGVDFQY |
| | | KG (SEQ ID No. 148) | (SEQ ID No. 149) |
| | V_{L} CDR1 | V_{L} CDR2 | V_{L} CDR3 |
| 2A6 | I: SGSVASHY | I: EDS | I: QSYYGNNQVL |
| | (SEQ ID No. 132) | (SEQ ID No. 133) | (SEQ ID No. 134) |
| | K: | K: EDSHRPS | K: QSYYGNNQVL |
| | VRSSGSVASHYVQ | (SEQ ID No. 157) | (SEQ ID No. 134) |
| | (SEQ ID No. 156) | | |
| 2B6 | I: RGYIASYY | I: EDT | I: QSYDDANHVI |
| | (SEQ ID No. 135) | (SEQ ID No. 136) | (SEQ ID No. 137) |
| | K: | K: EDTQRPS | K: QSYDDANHVI |
| | TRGRGYIASYYVQ | (SEQ ID No. 159) | (SEQ ID No. 137) |
| | (SEQ ID No. 158) | | |
| 2A6A | I: SGSVASHY | I: EDS | I: QSYYGNNQVL |
| | (SEQ ID No. 132) | (SEQ ID No. 133) | (SEQ ID No. 134) |
| | K: VRSSGSVASHYVQ | K: EDSHRPS | K: QSYYGNNQVL |
| | (SEQ ID No. 156) | (SEQ ID No. 157) | (SEQ ID No. 134) |
| 2A6Q | I: SGSVASHY | I: EDS | I: QSYYGNNQVL |
| | (SEQ ID No. 132) | (SEQ ID No. 133) | (SEQ ID No. 134) |
| | K: VRSSGSVASHYVQ | K: EDSHRPS | K: QSYYGNNQVL |
| | (SEQ ID No. 156) | (SEQ ID No. 157) | (SEQ ID No. 134) |
| BA | I: SGSVASHY | I: EDS | I: QSYYGNNQVL |
| | (SEQ ID No. 132) | (SEQ ID No. 133) | (SEQ ID No. 134) |
| | K: | K: EDSHRPS | K: QSYYGNNQVL |
| | VRSSGSVASHYVQ | (SEQ ID No. 157) | (SEQ ID No. 134) |
| | (SEQ ID No. 156) | | |

### Example 2: IFN-γ, CXCL9, CXCL10, and CXCL11 are Highly Expressed in the Skin Lesion of Vitiligo Patients

To gain insight into possible disease-causing pathways, we first performed cytokine profiling on the skin lesions of vitiligo patients and healthy control. We selected IFN-γ, CXCL9, CXCL10, CXCL11, IL13, IL15, IL17A, and IL18 based on the previous cohorts.

Materials and Methods: The high-sensitivity Meso Scale Discovery (MSD) electrochemiluminescence assay for the cytokine panel study was utilized to measure IFN-γ, CXCL9, CXCL10, CXCL11, IL-13, IL-15, IL-17A, IL18 in the skin suction blister interstitial fluid analyte. MSD plates were analyzed on the MS2400 imager (MSD). (Dabitao et al., 2011) The sample volume requirement for MSD is much less than conventional ELISA with higher sensitivity, enabling more cytokine to be studied in a single blister interstitial fluid analyte. (Hwang et al., 2019) The MSD assay was performed based on the manufacturer's instructions. All standards and samples were measured in a triplicate manner. Some cytokine panel results from the same patients in this study have been utilized in the clinical biomarker study for vitiligo activity and severity. (CY Ng, 2022)

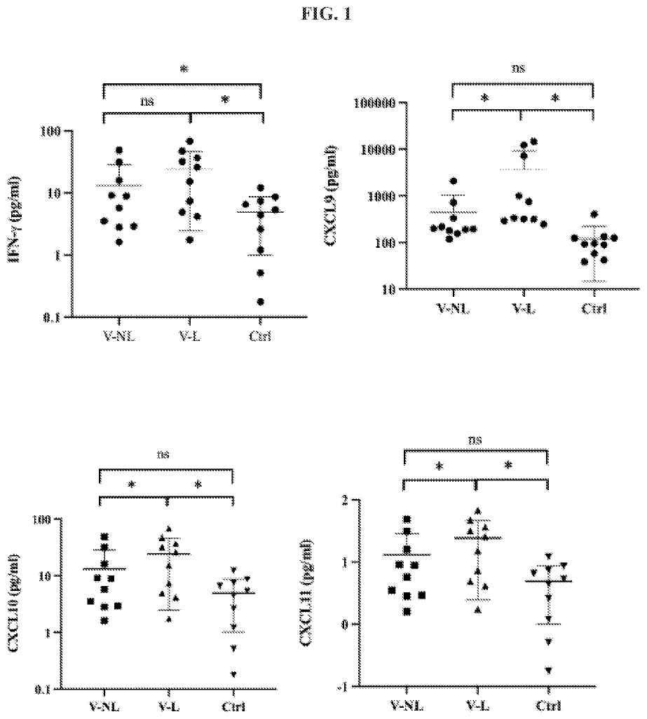

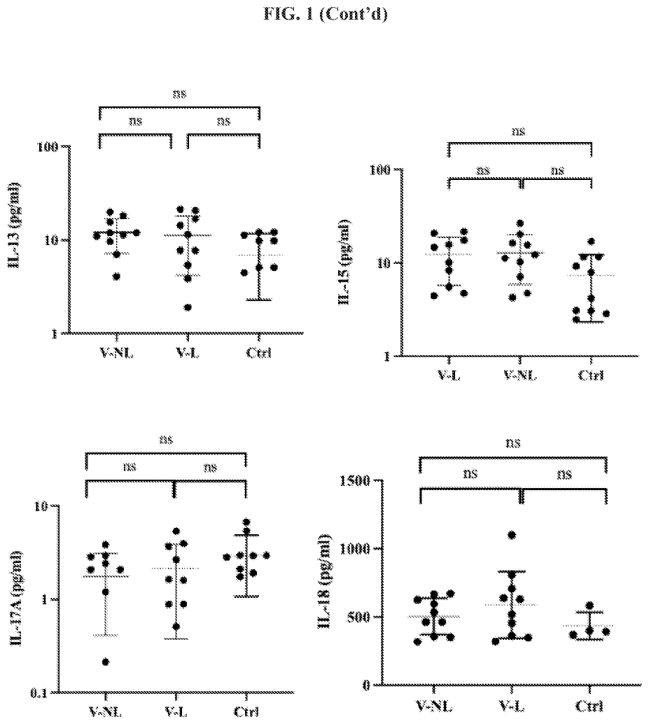

Results: As shown in FIG. 1, paired blister interstitial fluid from a lesion (V-L) and non-lesion (V-NL) skin were collected from thirty-two vitiligo patients and ten healthy volunteers (Ctrl) were collected and analyzed by a selected panel of cytokines by MSD immunoassays. The average age was 44.58±15.81 for vitiligo patients and 42.82±8.69 years for healthy controls. The male-to-female ratio in both healthy and vitiligo patients were 1:1. The BSA for vitiligo was 12% (±13.1%) and VES score was 9.02 (±9.822). IFN-γ and its' downstream cytokines CXCL9, CXCL10, and CXCL11 are highly expressed in vitiligo patients than of healthy controls. By way of comparison, no significant changes in IL-15, IL-17A, and IL-18 cytokines were observed.

Example 3: IFN-γ Directly Affects Cell Viability and Cell Morphological Change in Melanocytes

Functional study was performed to explore the pathogenic role of highly expressed cytokines-IFN-7, CXCL9, CXCL10, and CXCL11 to melanocytes.

Materials and methods: To examine the dose-dependency of IFN-γ inhibition of human primary melanocytes (HEMn-DP) cell viability, HEMn-DP cells were seeded into a 96-well plate at a concentration of 10,000 cells/well in 100 μL assay medium (M254 supplemented with 1× HMGS, 10 ng/mL PMA plus Penicillin-Streptomycin) at 37° C. for 5 hours. After seeding, 100 μL of IFN-γ serial dilutions were added, followed by incubation at 37° C. for 72 hrs. Then, cell proliferation was measured using CellTiter-Glo kit (Promega) according to the manufacturer's instructions. Luminescence was measured with an iD3 multimode reader (Molecular Devices), and data analyzed using nonlinear regression curve fit using GraphPad Prism7.

To determine the potency of 2A6Q, i.e., EI-001, HEMn-DP cells were seeded as previously described, and IFN-γ pre-incubated with various concentrations of EI-001 for 25 min in 100 μL of assay medium. After incubation, the EI-001 mixture was added to each well (final IFN-γ concentration is 30 ng/ml) and the melanocytes were cultured for additional 72 hrs at 37° C. and cell proliferation of the melanocytes was determined by luciferase activity using Celltiter Glo.

To determine cell viability of melanocyte after treatment with IFN-γ, CXCL9, CXCL10, CXCL11, at Day 2, and Day 7, the cells were collected and seeded into 96-well plates. After adhesion, cells were treated with IFN-γ (0, 10, 100 ng/ml) with different compounds: 2A6Q, CXCL9, CXCL10, CXCL11, ROS scavenger (NAC), necroptosis inhibitor (NEC-1), pyroptosis inhibitor (VX-765), apoptosis inhibitor (QVD-OPH), Ferroptosis inhibitor (Liproxstaitin-1, Ferrostatin-1, DFO). Cell viability was measured using a CellTiter-Glo luminescent cell viability assay kit (Promega) according to the manufacturer's protocol. The absorbance of the final reaction product was measured using a luminometer (SpectraMax® iD3 Multi-Mode Microplate Reader (Molecular Devices). After calculation, the viability of control cells was 100%, and all others were normalized to control and shown as relative cell viability (%). Treatment dosage N-acetylcysteine (NAC; 0.05 mM, 1 mM), Necrostatin-1 (NEC-1; 20 μM, 2 μM), Belnacasan (VX-765; 50 μg/mL, 5 μg/mL), Quinoline-Val Asp-Difluorophenoxymethylketone (QVD-OPH; 10 μM, 2 μM), Liproxstaitin-1 (10 μM, 5 μM, 1 μM, 0.05 μM), Ferrostatin-1 (5 μM, 1 μM), Deferoxamine (DFO; 10 μM, 5 M, 1 μM, 0.05 μM), 2A6Q (10 μg/mL), CXCL9 (10 ng/mL, 10 pg/mL), CXCL10 (10 ng/ml, 10 pg/mL), CXCL11(10 ng/mL, 10 pg/mL).

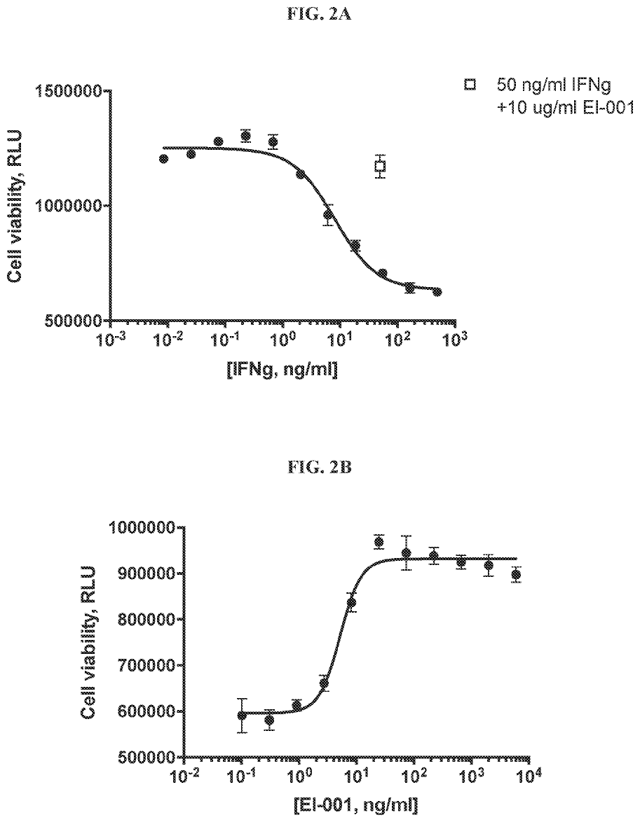

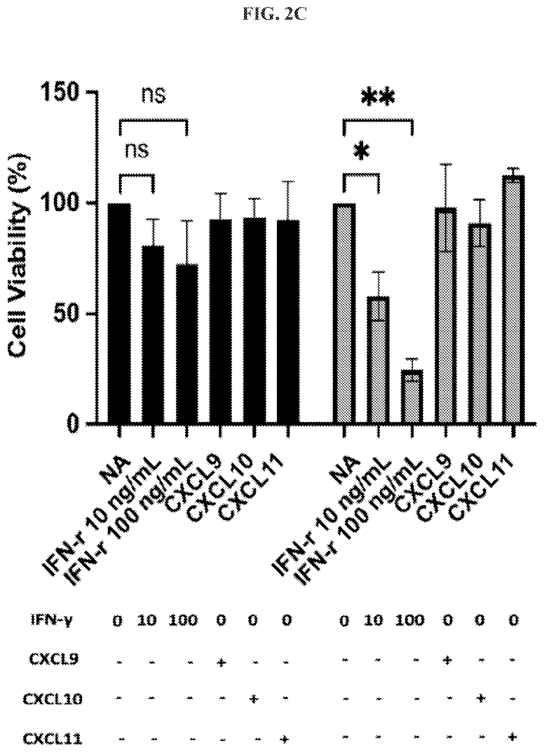

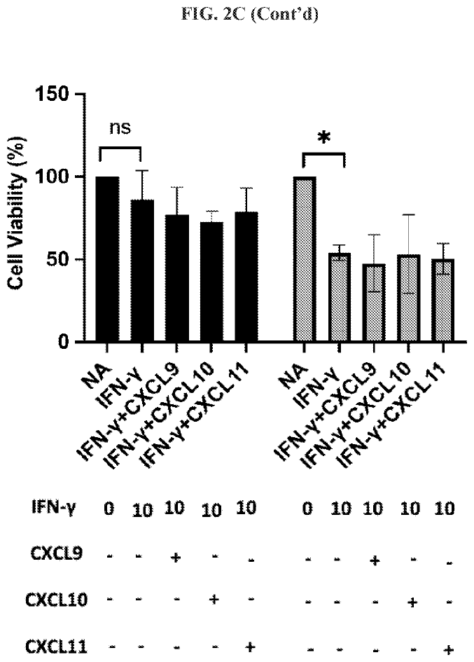

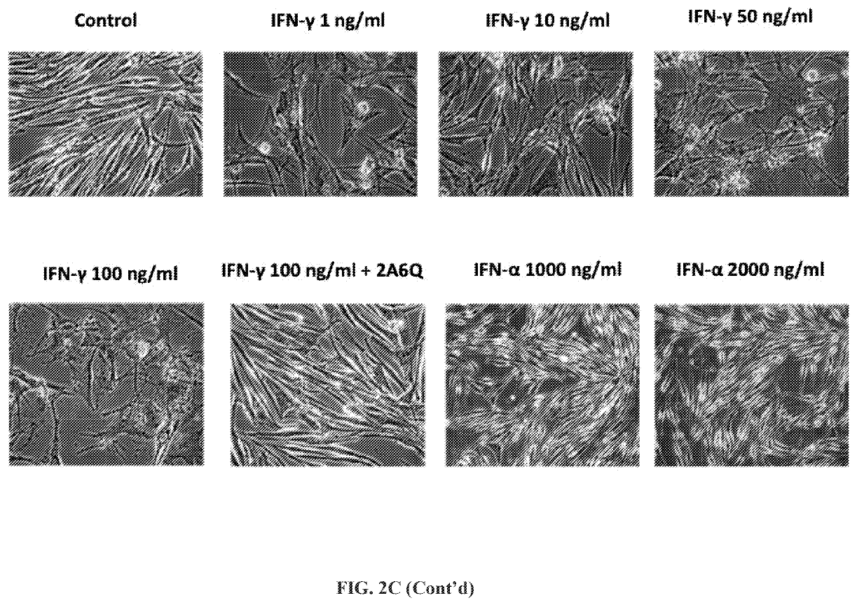

Results: As shown in the FIG. 2A, IFN-γ dose-dependently decreased viability of melanocytes. Various concentrations of recombinant human IFN-γ were added to human primary melanocytes. The calculated IC50 of IFN-γ causing 50% inhibition of melanocyte viability was 8.114 ng/mL. As shown in the FIG. 2B, EI-001 neutralized the proliferation inhibition effect of IFN-γ and restored cell viability. The calculated EC50 of EI-001 was 5.214 ng/ml.

As shown in the FIG. 2C, IFN-γ but not CXCL9, CXCL10, or CXCL11 significantly decreases the cell viability of melanocytes at day 7 of treatment. The synergistic effects of IFN-γ, CXCL9, CXCL10, and/or CXCL11 on the cell viability of melanocytes were subsequently investigated. In particularly, the CXCL10 have been showed to enhance the IFN-γ-mediated cytotoxicity in melanocyte. Intriguingly, a synergistic effect was not found between these cytokines. Therefore, IFN-γ per se is the most critical cytokine for melanocyte cell viability. IFN-γ directly affects cell viability and cell morphological change in melanocytes.

Type I IFNs and IFN-γ share an overlapping but non-identical pathways through JAK/STAT pathways. In the cell morphology assay, IFN-γ but not IFN-α was found to contributes to morphology change in melanocyte, with loss of dendrite and ballooning of the melanocyte cell cytoplasm and the effect of IFN-γ was dose-dependent and present as low as 1 ng/ml in concentration, but not IFN-α.

### Example 4: IFN-γ Disrupts Melanocyte Melanogenesis

To investigate the effect of IFN-γ on melanogenesis, melanocytes were treated with IFN-γ 100 ng/mL with paired control for transcriptome analysis.

Materials and methods: To examine the dose-dependency of IFN-γ inhibition of HEMn-DP cell melanogenesis, HEMn-DP cells were seeded into a 24-well plate at a concentration of 200,000 cells/well in 1 mL assay medium (M254 supplemented with 1× HMGS, 10 ng/mL PMA plus Penicillin-Streptomycin) with serial dilutions of IFN-γ, followed by incubation at 37° C. for 7 days. The cell assay medium was replaced by fresh medium with serially titrated concentrations of IFN-γ on day 3 and day 5. On day 7, cells were first washed with PBS twice, lysed in 250 μL of 1N NaOH/10% DMSO solution, then incubated at 80° C. for 1 hr. After lysis, each lysate was transfer to 96 well plate, and absorbance of melanin was then measured at 470 nM. The relative melanin quantity was normalized with cell viability of each sample which was measured by CellTiter-Glo kit, and data were analyzed using nonlinear regression curve fit using GraphPad Prism7. To determine the potency of EI-001, HEMn-DP cells were seeded as previously described, and IFN-γ pre-incubated with indicated amounts of EI-001 for 25 min in 1 mL of assay medium. After incubation, the mixture was added to each well (final IFN-γ concentration is 20 ng/ml). Melanin content of melanocytes was determined as previously described.

Total RNA was extracted from melanocytes with an RNA purification kit (Zymo Research, r2050). After RNA samples were extracted from primary melanocytes and melanocytes cell line (Gibco), the samples were sent to Biotools Co., Ltd Taiwan for RNA sequence. The RNA quality was examined by Agilent Technologies 2100 Bio-analyzer with an RNA integrity value greater than 8. After rRNA depletion (Epicenter), RNA fragmentation, and library preparation (Illumina), the constructed libraries were then performed 150 bp paired-end sequencing by an Illumina NovaSeq6000 sequencer. The results from each sample were aligned to the GRCh38 genome by the HISAT2 software and analyzed by Biotools Co., Ltd Taiwan.

Cells were washed in PBS and lysed in RIPA lysis buffer with proteases inhibitor and Phenylmethanesulfonyl fluoride (PMSF; Sigma-Aldrich, P7626). The total cell lysates were sonicated and centrifuged at 4° C., 13000 rpm for 30 min. Protein concentration was quantified using the Bradford assay. The total cell lysates were separated by SDS-PAGE and transferred to a PVDF membrane. The membrane was blocked with 5% BSA or 5% milk in TBST for 1 hour at room temperature and incubated with primary antibodies overnight at 4° C. and HRP-conjugated secondary antibodies for hour at room temperature. The protein was visualized by Thermo ibright1500. Primary antibodies were follows: anti-human SLC7A11 (CST, 12691), anti-human SLC3A2 (CST, 13180), anti-IRF1 (CST, 8478), anti-MITF (CST, 12590), and anti-GAPDH (Abcam, ab8245).

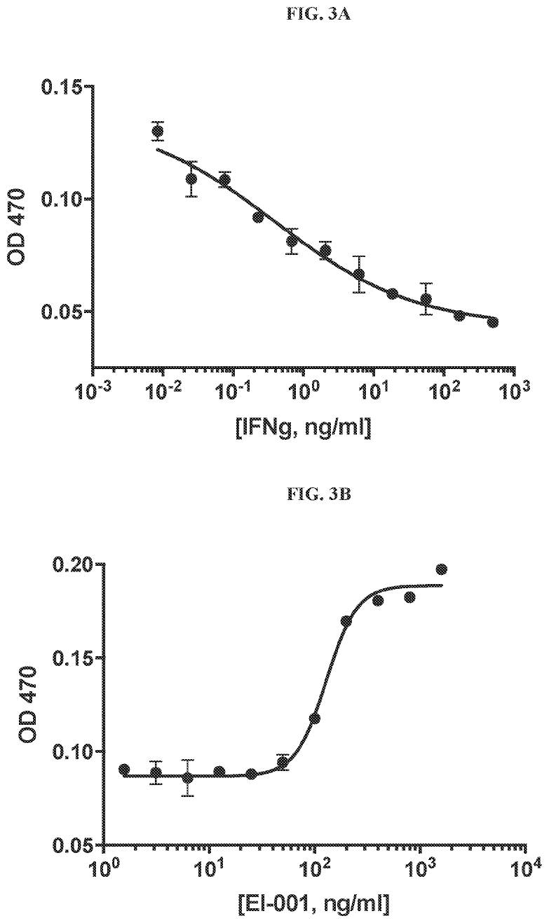

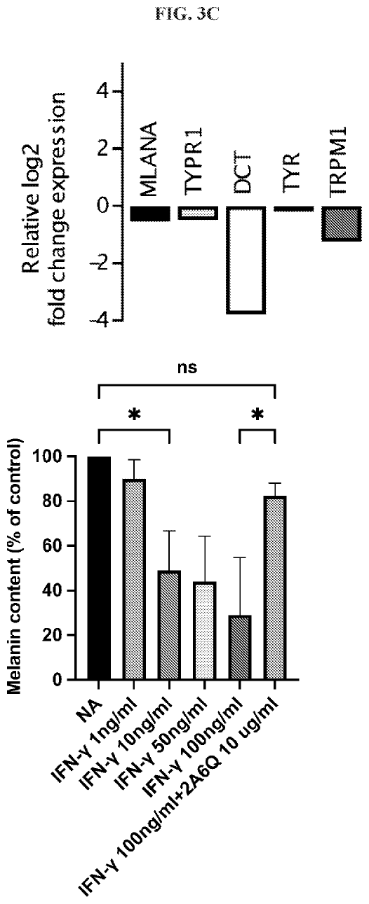

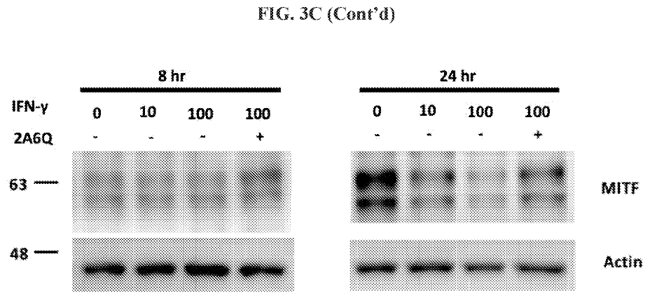

Results: As shown in FIG. 3A, IFN-γ dose-dependently decreased melanogenesis of melanocytes. The calculated IC50 of IFN-γ causing 50% inhibition of melanocyte melanogenesis was 0.392 ng/mL. As shown in the FIG. 3B, EI-001 neutralized the melanogenesis inhibition effect of IFN-γ and restored melanogenesis of the melanocyte. The calculated EC50 of IFN-γ to restore 50% inhibition of melanocyte melanogenesis was 129.1 ng/mL. As shown in FIG. 3C, IFN-γ induced downregulation of melanin regulators (MITF, MLANA, TYRP1, DCTT, TYR, TRPM1) in primary melanocyte. To further confirm the finding, immunoblotting of MITF was performed in melanocytes, a key regulator for melanogenesis, at 8 hrs and 24 hrs after treatment with various doses of IFN-γ. The results show that the IFN-γ dose-dependently downregulates the expression of MITF in melanocytes at 24 hrs of treatment. Furthermore, spectrophotometer further confirmed a decrease in melanin content in a dose-dependent manner with IFN-γ treatment, which can be rescued by addition of 2A6Q.

### Example 5: EI-001 Inhibited CXCR3 Expression on CD4+and CD8+T Cells

This example illustrates EI-001 (2A6Q), an IFN-γ neutralizing antibody, inhibited the expression of CXCR3 on CD4+ and CD8+ T cells during T cell activation.

Materials and methods: PBMCs from healthy adult donors were isolated from whole blood using Ficoll-Paque density gradient centrifugation. PBMCs were cultured in a humidified 37° C. incubator for 24 hours in RPMI-1640 culture medium supplemented with 5% human serum, and standard antibiotics, 2-ME and HEPES. PBMCs were then seeded at 1×10⁶ cells/well with 1 mL fresh culture medium/well in a 24-well plate and stimulated by mixing cells with human T cell TransAct (1:200). Antibodies indicated in the Results were then added and mixed well. After 2 days of culture, PBMCs were collected, washed with PBS three times, re-suspended in culture medium and re-cultured with indicated antibodies for an additional 2 days.

PBMCs were treated as required (see above) and then harvested for flow cytometry antibody staining, using anti-human CD4, anti-human CD8, anti-human CD56 and anti-human CXCR3 antibodies for 30 minutes on ice in the dark. PBMCs were then washed twice with FACS buffer and the expression of CXCR3 determined by flow cytometry using an Attune NxT flow cytometry. Flow cytometry data analysis was performed using FlowJo software.

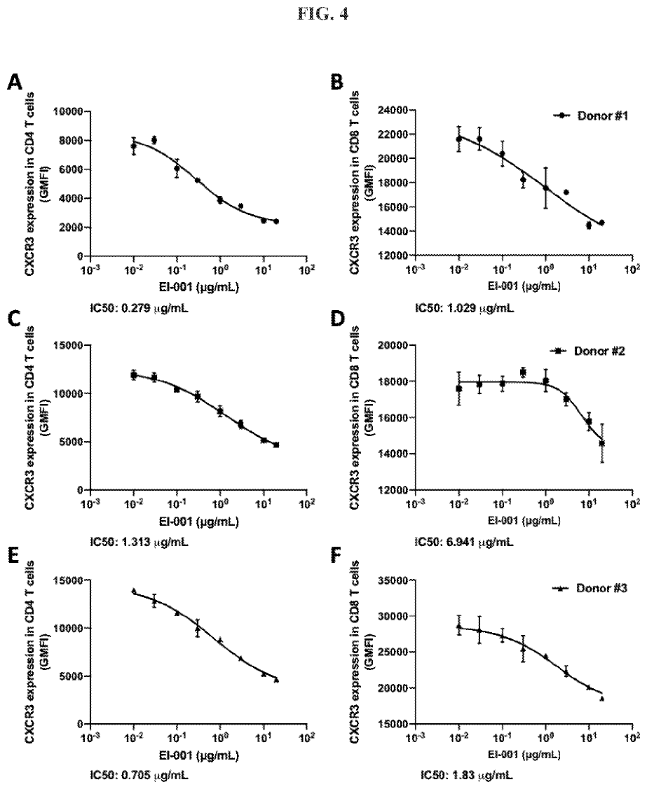

Results: As shown in FIG. 4, to understand the inhibitory capacity of EI-001 in CXCR3 expression on T cells, EI-001 (2A6Q) was added into culture medium during activation of PBMCs. The expression of CXCR3 on CD4+ T cells and CD8+ T cells was determined by flow cytometry. The IC50 of EI-001 to inhibit CXCR3 expression on CD4+ T cells was 0.279-1.313 μg/mL, and the IC50 of that on CD8+ T cells was 1.029-6.941 μg/mL (FIG. 4). This data supports the conclusion that neutralization of IFN-γ by EI-001 (2A6Q) inhibits CXCR3 expression on CD4 and CD8+ T cells during T activation.

### Example 6: IFN-γ Induces Early-Phase Apoptosis in Melanocyte

This example illustrates IFN-γ induces early-phase apoptosis in melanocyte, which can be rescued by 2A6Q.

Materials and methods: Melanocyte cell apoptosis was detected with Annexin V (BD, 550474) and 7AAD (1 mg/mL, Sigma A9400-1 MG). The cells were detached and resuspended with 100 mL binding buffer (BD, 556454) containing Annexin V and 7AAD for 15 min at room temperature in the dark. Before analysis, cells were added to a 300 mL binding buffer and immediately analyzed a on a flow cytometer (Verse BD).

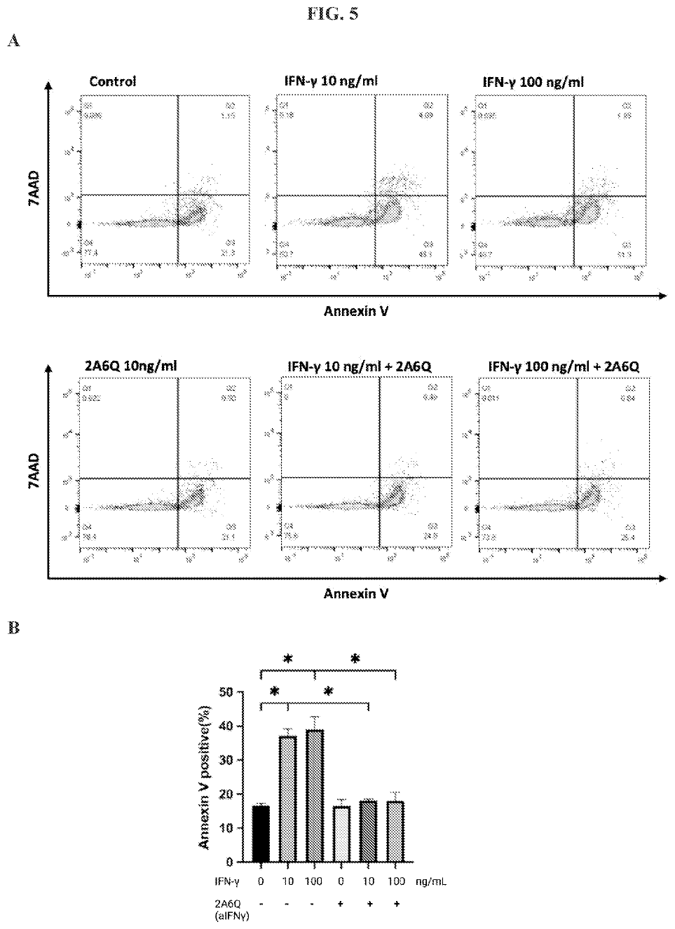

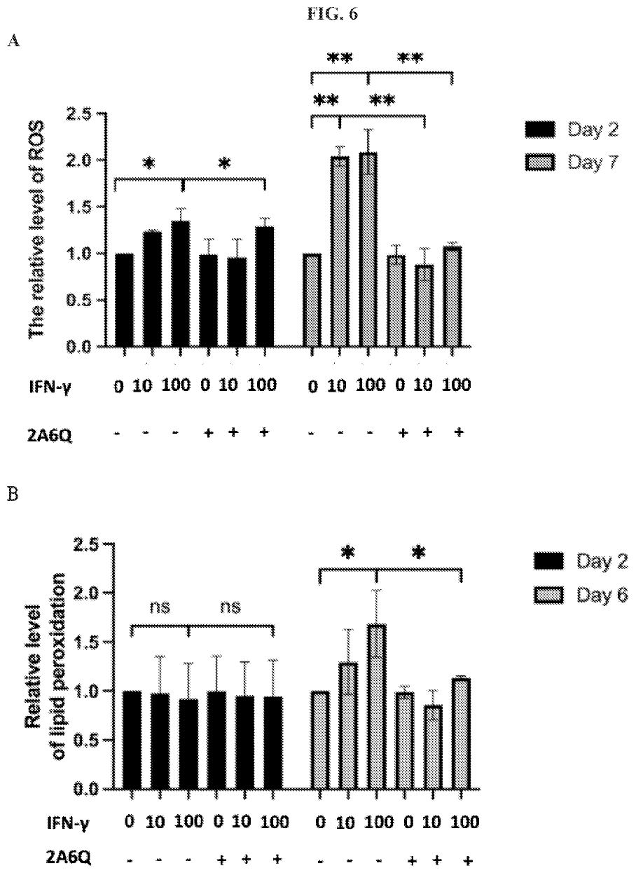

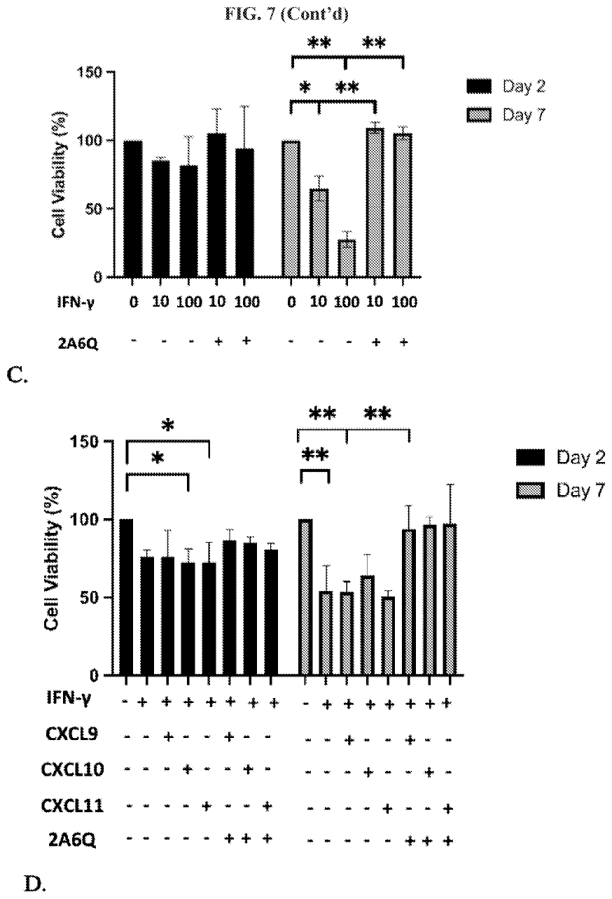

Results: FIG. 5 depicts that IFN-γ induced early phase apoptosis in melanocytes, as demonstrated by Annexin V+ in a dose-dependent manner and 2A6Q managed to reverse the apoptosis. However, pan-caspase inhibitor was unable to rescue IFN-γ induced cell death in melanocytes, indicating, in addition to induce apoptosis, other alternative pathways contributing to the cell loss in the melanocyte. (FIG. 7A)

### Example 7: IFN-γ Increases ROS Lipid Peroxidation and Induces Ferroptosis Cell Death in Melanocyte

IFN-γ has been reported to cause ferroptosis and lipid peroxidation during melanoma cancer immunotherapy, contributing to the release of the damage-associated molecular pattern (DAMP) in melanoma tumors during cancer immunotherapy. Similar studies in normal melanocytes have not been performed.

Materials and methods: For intracellular ROS measurement, melanocytes were washed with PBS and incubated with 20 μM CM-H2DCFDA (Invitrogen) for 30 min at 37° C. in the dark. The cells were resuspended with PBS and examined with flow cytometry at an excitation wavelength of 488 nm and an emission wavelength of 530 nm; the data were analyzed with FlowJo.

For lipid peroxidation examination, melanocyte cells were seeded in a 6-well plate and treated with IFN-γ the next day. Melanocytes were washed with PBS and incubated with Hanks balanced salt solution (HBSS, Gibco) containing 5 μM BODIPY 581/591 C11 (Invitrogen, D3861) and incubated for 15 minutes at 37° C. in a tissue culture incubator. The cells were resuspended with fresh HBSS and examined with flow cytometry; the data were analyzed with FlowJo.

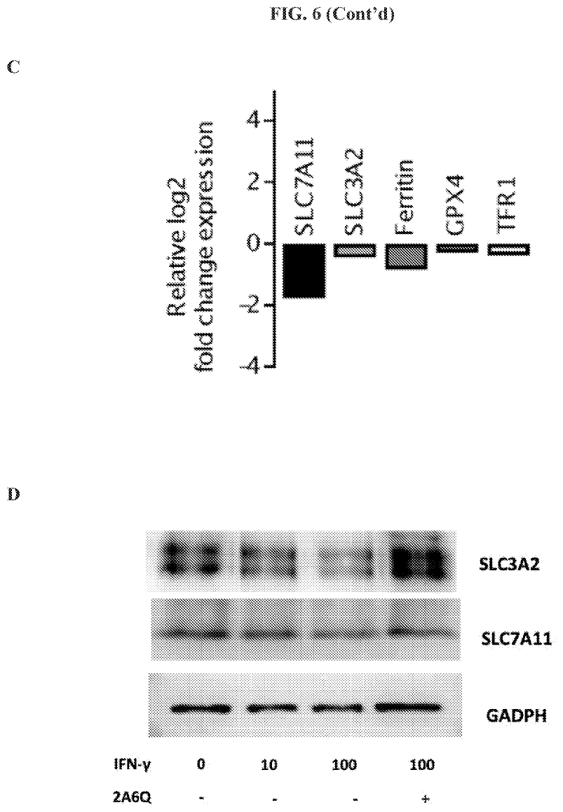

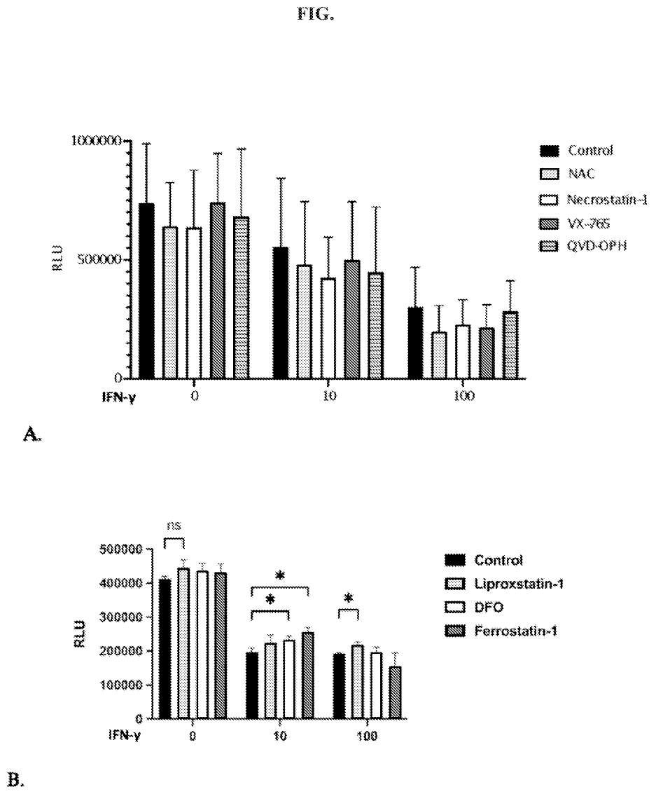

Results: As shown in FIG. 6A, IFN-γ significantly elevated intracellular ROS at in both 10 and 100 ng/mL IFN-γ on day 7, measured by the 20 μM CMH2DCFDA. In addition, the lipid peroxidation level was increased in melanocytes treated with IFN-γ in a dose-dependent manner on Day 6. (FIG. 6B) RNA sequencing analysis of melanocyte transcriptomics expression for ferroptosis marker shows downregulation of SLC7A11, SLC3A2, Ferritin, GPX4 and TFR1 after incubation with 24 hrs IFN-γ. (FIG. 6C) Immunoblots of SLC7A11 after 48 hours incubation with IFN-γ also show that IFN-γ dose-dependently downregulated the expression of SLC7A11 in melanocytes, indicating the ferroptosis process. (FIG. 6D)

### Example 8: Human Anti-IFN-γ Monoclonal Antibody (2A6Q) Reverses the Melanocyte Cell Death and Melanogenesis of Melanocyte

Melanocyte lost is a hallmark of vitiligo; therefore, we aimed to address the effect of ROS scavenger and other anti-death molecules to rescue the IFN-γ-mediated melanocyte lost.

Results: As shown in FIG. 7, N-acetyl cysteine (NAC) was supplied to the medium as the ROS scavenger but did not find a rescue effect with NAC. Intriguingly, 2A6Q reverses the ROS in melanocytes induced by IFN-γ. To study induced melanocyte cell death other than apoptosis, the cell viability study was repeated in melanocytes with IFN-γ for seven days with necroptosis inhibitor (NEC-1), pyroptosis inhibitor (VX-765), apoptosis inhibitor (QVDOPH pan-caspase inhibitor), and 2A6Q. (FIG. 7A) Ferroptosis inhibitor Ferrostatin-1, DFO, and Liproxstatin-1 were utilized to study IFN-γ induced ferroptosis in melanocytes. The results showed that Liproxstatin-1 1 μM can partially rescue IFN-γ induced melanocyte cell loss. In comparison, the rescue effect of 2A6Q is more profound than all other cell death inhibitors. (FIG. 7B) Moreover, 2A6Q also reverses the downregulation of MITF and decrease in melanin content FIG. 3C), early phase apoptosis (FIG. 5B), cellular oxidative stress (FIG. 6A), lipid peroxidation (FIG. 6B), and reverses ferroptosis as demonstrated by SLC7A11 in melanocytes. (FIG. 6D)

### Example 9: 2A6Q was Well-Tolerated at All Dose Levels and exhibits Nearly No Side Effects Comparing to NI-0501

This example illustrates EI-001 (2A6Q), an IFN-γ neutralizing antibody, when assessed potential toxicity after repeated intravenous infusion of EI001 for 4 weeks, was well-tolerated and showed better side effect profile comparing to another anti-IFN-γ antibody (NI-0501).

Materials and methods: A total of forty Cynomolgus monkeys (20 females and 20 males) were assigned into 4 groups with 5 animals/gender/group and given EI-001 (2A6Q) (10, 30 and 100 mg/kg) or vehicle (Formulation Buffer) in a volume of 10 mL/kg via 30-min intravenous infusion, once weekly for a total of 5 doses, followed by a 6-week recovery phase. All animals were scheduled to be necropsied at the end of main phase (on Day 30) and at the end of recovery phase (on Day 72). During the study, the following parameters were evaluated: clinical observations, ophthalmology, body weight, food consumption, body temperature (rectal temperature), clinical pathology (hematology, coagulation, plasma chemistry, lymphocyte immunophenotype and urinalysis), gross pathology, organ weight and histopathology. toxicokinetics, immunogenicity, local irritation assessment, cardiovascular (ECG, body surface temperature, blood pressure), respiratory and central nervous system safety pharmacology assessments were conducted concurrently. Toxicokinetics samples were collected on pre-dose, immediately, 1 hr, 4 hrs, 8 hrs, 24 hrs, 72 hrs, 120 hrs and 168 hrs post the first and fourth doses, pre-dose and immediately post the third dose. The immunogenicity samples were collected on pre-dose on Days 1, 15, 22, and Days 57, 71.

Results: As shown in Table 3, all animals treated by EI-001 (2A6Q) survived to their scheduled necropsy. No EI-001 (2A6Q) related changes in clinical observations, ophthalmology, body weights, food consumptions, local irritation, body temperature (rectal temperature), safety pharmacology evaluation (cardiovascular parameters, respiratory and central nervous system), clinical pathology (hematology, coagulation, plasma chemistry and urinalysis parameters), lymphocyte immunophenotype, gross pathology or organ weights were noted in this study. Furthermore, the only detected changes related to EI-001 (2A6Q) administration were seen on a microscopic level. When compared to prior toxicology studies using an anti-IFN-γ antibody NI-0501 (NDA/BLA Multi-Disciplinary Review and Evaluation application number: 761107; www.accessdata.fda.gov/drugsatfda_docs/nda/2018/761107Orig1s000MultidisciplineR.pdf), treatment with EI-001 (2A6Q) did not increase either mortality rates or susceptibility to infections among the treated monkeys. Moreover, treatment with EI-001 (2A6Q) did not induce observable symptoms of infection. These findings suggest that EI-001 (2A6Q) does not induce any signs of infection. The overall results indicate that EI-001 (2A6Q) treatment could be a safer and potentially more effective approach to neutralize IFN-γ. Therefore, EI-001 (2A6Q) could have better therapeutic efficacy in the treatment of diseases induced by IFN-γ, such as vitiligo.

TABLE 3

| | | | Dose/animal | |
|---|---|---|---|---|
| Antibody | Type | GLP | species | Key Study Findings |
| NI-0501 | General | Yes | 0, 10, | An increase in |
| (Emapa- | toxicology | | 100 or 200 | susceptibility |
| lumab) | | | mg/kg | to infection as |
| | | | weekly | consequence of an |
| | | | Cynomolgus | exaggerated |
| | | | monkeys | immunomodulating |
| | | | | (pharmacological) effect |
| | | | | of NI-0501 can' t |
| | | | | be ruled out. |
| | | | | There were mortalities |
| | | | | at all dose groups. From |
| | | | | 18 monkeys, total of |
| | | | | 7 monkeys were early |
| | | | | sacrificed because of |
| | | | | severe clinical |
| | | | | signs. GI tract |
| | | | | infection was confirmed |
| | | | | as caused by Shigella sp. |
| | | | | and Campylobacter sp. |
| | | | | It seems likely that the |
| | | | | pharmacological effect of |
| | | | | IFN-γ inhibition results in |
| | | | | these opportunistic |
| | | | | infections. |
| | | | | Associated clinical signs |
| | | | | included liquid feces, |
| | | | | dehydration, and moribund |
| | | | | sacrifices of seven |
| | | | | NI-0501-treated monkeys; |
| | | | | manifestation of the |
| | | | | intestinal infections |
| | | | | included changes in |
| | | | | clinical pathology and |
| | | | | histopathology. |
| EI-001 | General | Yes | 0, 10, | No significant EI- |
| (2A6Q) | toxicology | | 30 or 100 | 001(2A6Q)-related |
| | | | mg/kg | changes in mortality, |
| | | | weekly | clinical observations, |
| | | | Cynomolgus | ophthalmology, body |
| | | | monkeys | weight, food consumption, |
| | | | | body temperature, local |
| | | | | irritation assessment, |
| | | | | cardiovascular and |
| | | | | respiratory system safety |
| | | | | pharmacology, central |
| | | | | nervous system safety |
| | | | | pharmacology, |
| | | | | hematology, coagulation, |
| | | | | plasma chemistry, |
| | | | | lymphocyte |
| | | | | immunophenotype, |
| | | | | urinalysis, gross pathology, |
| | | | | organ weights, |
| | | | | and most of microscopic |
| | | | | pathology findings. |

In summary, we demonstrated that single agent to block the apoptosis, necroptosis, ferroptosis and ROS production failed to protect the melanocyte from IFN-γ-mediated cytotoxicity. These observations suggest the cytotoxicity effect of IFN-γ is mediated through multiple pathways. In contrast to blocking selected cell death pathways, our in vitro study results support the rescue effect of 2A6Q in IFN-γ induced cell death, oxidative stress, and loss of function in melanocytes by interrupting the IFN-γ signaling, which is a potential therapeutic option for vitiligo. Furthermore, the in vivo toxicology results show that EI-001 (2A6Q) treatment does not cause any significant drug adverse events. As such, EI-001 (2A6Q) could be a safe and promising therapeutic strategy for vitiligo.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims. Additional embodiments of the invention are set forth in the following claims.

1. A method for treating vitiligo, comprising administering to a subject in need thereof a composition comprising a therapeutically effective amount of an IFN-γ antibody and a pharmaceutically acceptable carrier.

2. The method of claim 1, wherein the IFN-γ antibody modulates a biological function of IFN-γ.

3. The method of claim 1, wherein the IFN-γ antibody: (a) neutralizes IFN-γ; or (b) antibody inhibits, decreases, and/or fully blocks the signaling activity of IFN-γ.

4. (canceled)

5. The method of claim 3, wherein the IFN-γ antibody decreases IFN-γ signaling activity by pro-human IFN-γ, mature-human IFN-γ, or truncated-human IFN-γ.

6. The method of claim 1, wherein the IFN-γ antibody reduces: (a) one or more of: IFN-γ-dependent cytokine production; IFN-γ-dependent T cell dysfunction, IFN-γ-dependent immune tolerance, and IFN-γ-dependent inflammation; or (b) expression of IFN-γ, CXCL9, CXCL10 and/or CXCL11 in melanocytes.

7. (canceled)

8. The method of claim 1, wherein the IFN-γ antibody increases IL-2 production and/or cell proliferation of SEB-stimulated human PBMCs by at least 1,2-fold, 1.3-fold, 1.4-fold, 1.5-fold, 1.6-fold, 1.7-fold, 1.8-fold, at least 1.9-fold, at least 2-fold, at least 2.1-fold, or at least 2.20-fold.

9. The method of claim 1, wherein the IFN-γ antibody comprises:
a VH region having a CDR-H1 amino acid sequence selected from SEQ ID NO: 120 or 123, a CDR-H2 amino acid sequence selected from SEQ ID NO: 121 or 124, and a CDR-H3 amino acid sequence selected from SEQ ID NO: 122 or 125; and
a VL region comprising a CDR-L1 amino acid sequence selected from SEQ ID NO: 132 or 135, a CDR-L2 amino acid sequence selected from SEQ ID NO: 133 or 136, and a CDR-L3 amino acid sequence selected from SEQ ID NO: 134 or 137.

10. The method of claim 1, wherein the IFN-γ antibody comprises:
(a) a VH region having a CDR-H1 amino acid sequence of SEQ ID NO: 120, a CDR-H2 amino acid sequence of SEQ ID NO: 121, and a CDR-H3 amino acid sequence of SEQ ID NO: 122, and a VL region comprising a CDR-L1 amino acid sequence of SEQ ID NO: 132, a CDR-L2 amino acid sequence of SEQ ID NO: 133, and a CDR-L3 amino acid sequence of SEQ ID NO: 134;
(b) a VH region having a CDR-H1 amino acid sequence of SEQ ID NO: 123, a CDR-H2 amino acid sequence of SEQ ID NO: 124, and a CDR-H3 amino acid sequence of SEQ ID NO: 125, and a VL region comprising a CDR-L1 amino acid sequence of SEQ ID NO: 135, a CDR-L2 amino acid sequence of SEQ ID NO: 136, and a CDR-L3 amino acid sequence of SEQ ID NO: 137; or
(c) a VH region having a CDR-H1 amino acid sequence of SEQ ID NO: 123, a CDR-H2 amino acid sequence of SEQ ID NO: 124, and a CDR-H3 amino acid sequence of SEQ ID NO: 125, and a VL region comprising a CDR-L1 amino acid sequence of SEQ ID NO: 132, a CDR-L2 amino acid sequence of SEQ ID NO: 133, and a CDR-L3 amino acid sequence of SEQ ID NO: 134.

11. The method of claim 1, wherein the IFN-γ antibody comprises:
(a) a VH region comprising an amino acid sequence having at least 90% identity to SEQ ID NO: 109, 110, 164, or 165; and
(b) a VL region comprising an amino acid sequence having at least 90% identity to SEQ ID NO: 113, or 114.

12. The method of claim 1, wherein the IFN-γ antibody comprises:
(a) a VH region amino acid sequence of SEQ ID NO: 109, and a VL region amino acid sequence of SEQ ID NO: 113;
(b) a VH region amino acid sequence of SEQ ID NO: 110, and a VL region amino acid sequence of SEQ ID NO: 114;
(c) a VH region amino acid sequence of SEQ ID NO: 109, and a VL region amino acid sequence of SEQ ID NO: 114;
(d) a VH region amino acid sequence of SEQ ID NO: 110, and a VL region amino acid sequence of SEQ ID NO: 113;
(e) a VH region amino acid sequence of SEQ ID NO: 164, and a VL region amino acid sequence of SEQ ID NO: 113; or
(f) a VH region amino acid sequence of SEQ ID NO: 165, and a VL region amino acid sequence of SEQ ID NO: 113.

13. The method of claim 10, wherein the IFN-γ antibody comprises:
(a) a heavy chain amino acid sequence having at least 90% identity to SEQ ID NO: 183, 185, 187, or 189; and
(b) a light chain amino acid sequence having at least 90% identity to SEQ ID NO: 184, or 186.

14. The method of claim 1, wherein the IFN-γ antibody comprises:
(a) a heavy chain amino acid sequence of SEQ ID NO: 183, and a light chain amino acid sequence of SEQ ID NO: 184;
(b) a heavy chain amino acid sequence of SEQ ID NO: 185, and a light chain amino acid sequence of SEQ ID NO: 186;
(c) a heavy chain amino acid sequence of SEQ ID NO: 183, and a light chain amino acid sequence of SEQ ID NO: 186;
(d) a heavy chain amino acid sequence of SEQ ID NO: 185, and a light chain amino acid sequence of SEQ ID NO: 184;
(e) a heavy chain amino acid sequence of SEQ ID NO: 187, and a light chain amino acid sequence of SEQ ID NO: 184; or
(f) a heavy chain amino acid sequence of SEQ ID NO: 189, and a light chain amino acid sequence of SEQ ID NO: 184.

15. The method of claim 10, wherein the VH region of the IFN-γ antibody further comprises an amino acid substitution selected from N76A and N76Q.

16. The method of claim 1, wherein the IFN-γ antibody is an antibody selected from the group consisting of 2A6, 2B6, 2A6A, 2A6Q, AB, BA, AMG811, NI-0501 and Fontolizumab.

17. The method of claim 17, wherein an infection or a drug induced adverse effect was not induced by 2A6Q in an in vivo toxicology assay.

18. The method of claim 1, wherein the IFN-γ antibody is an immunoglobulin molecule, an Fv, a disulfide linked Fv, a monoclonal antibody, an scFv, a chimeric antibody, a single domain antibody, a CDR-grafted antibody, a diabody, a human antibody, a humanized antibody, a multispecific antibody, an Fab, a dual specific antibody, an Fab′ fragment, a bispecific antibody, an F(ab′)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody, a dAb fragment, an isolated complementarity determining region (CDR), or a single chain antibody.

19. The method of claim 1, wherein administering to the subject is by at least one mode selected from the group consisting of: parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

20. The method of claim 1, wherein the subject in need is a patient with vitiligo.

21. The method of claim 1, wherein the composition is administered to the subject more than once a day, at least once a day, at least once a week, or at least once a month.

22. The method of claim 1, further comprising administering at least one additional therapeutic agent.

23. The method of claim 22, wherein the additional therapeutic agent is administered to the subject in need before administration of the composition, after administration of the composition, and/or at the same time as the composition.

24. The method of claim 1, wherein the IFN-γ antibody binds to: (a) human IFN-γ with a binding affinity of 1×10⁻⁸ M or less, 1×10⁻⁹ M or less, 1×10⁻¹⁰ M or less, or 1×10⁻¹¹ M or less; (b) rhesus macaque IFN-γ or cynomolgus monkey IFN-γ with a binding affinity of 1×10⁻⁸ M or less, 1×10⁻⁹ M or less, 1×10⁻¹⁰ M or less, or 1×10⁻¹¹ M or less; or human IFN-γ and to cynomolgus monkey IFN-γ with a binding affinity of 1×10⁻⁸ M or less, 1×10⁻⁹ M or less, 1×10⁻¹⁰ M or less, or 1×10⁻¹¹ M or less.

25. (canceled)

26. (canceled)

27. (canceled)

28. (canceled)

29. (canceled)

30. (canceled)

31. (canceled)

32. (canceled)

33. (canceled)

34. A composition comprising a therapeutically effective amount of an IFN-γ antibody and a pharmaceutically acceptable carrier for use in treating vitiligo in a subject in need thereof.

35. The composition of claim 34, wherein the IFN-γ antibody is an antibody selected from the group consisting of 2A6, 2B6, 2A6A, 2A6Q, AB, BA, AMG811, NI-0501 and Fontolizumab.
